SEQUENCE LISTING (70 sequences)

SEQ ID NO: 1 - protein, 8 aa, Artificial Sequence
  Description of Artificial Sequence Synthetic formula peptide n=0 and m=1
  MOD_RES (1): Hydrophobic amino acid
  MOD_RES (2)..(3): Variable amino acid
  MOD_RES (4): Hydrophobic amino acid
  MOD_RES (5): Small amino acid residue
  MOD_RES (6): Negatively charged amino acid
  MOD_RES (7): Variable amino acid
  MOD_RES (8): Hydrophobic amino acid
  see specification as filed for detailed description of substitutions and preferred embodiments
XXXXXXXX

SEQ ID NO: 2 - protein, 12 aa, Artificial Sequence
  Description of Artificial Sequence Synthetic formula peptide n=1 and m=1
  MOD_RES (1): Hydrophobic amino acid
  MOD_RES (2): Small amino acid
  MOD_RES (3)..(4): Variable amino acid
  MOD_RES (5): Hydrophobic amino acid
  MOD_RES (6)..(7): Variable amino acid
  MOD_RES (8): Hydrophobic amino acid
  MOD_RES (9): Small amino acid
  MOD_RES (10): Negatively charged amino acid
  MOD_RES (11): Variable amino acid
  MOD_RES (12): Hydrophobic amino acid
  see specification as filed for detailed description of substitutions and preferred embodiments
XXXXXXXXXXXX

SEQ ID NO: 3 - protein, 8 aa, Artificial Sequence
  Description of Artificial Sequence Synthetic formula peptide n=1 and m=0
  MOD_RES (1): Hydrophobic amino acid
  MOD_RES (2): Small amino acid
  MOD_RES (3)..(4): Variable amino acid
  MOD_RES (5): Hydrophobic amino acid
  MOD_RES (6)..(7): Variable amino acid
  MOD_RES (8): Hydrophobic amino acid
  see specification as filed for detailed description of substitutions and preferred embodiments
XXXXXXXX

SEQ ID NO: 4 - protein, 9 aa, Artificial Sequence
  Description of Artificial Sequence Synthetic formula peptide m=0
  MOD_RES (1): Variable amino acid and its side chain is linked to position 8 by a linker
  MOD_RES (2): Hydrophobic amino acid
  MOD_RES (3): Small amino acid
  MOD_RES (4)..(5): Variable amino acid
  MOD_RES (6): Hydrophobic amino acid
  MOD_RES (7): Variable amino acid
  MOD_RES (8): Variable amino acid and its side chain is linked to position 1 by a linker
  MOD_RES (9): Hydrophobic amino acid
  see specification as filed for detailed description of substitutions and preferred embodiments
XXXXXXXXX

SEQ ID NO: 5 - protein, 13 aa, Artificial Sequence
  Description of Artificial Sequence Synthetic formula peptide m=1
  MOD_RES (1): Variable amino acid and its side chain is linked to position 8 by a linker
  MOD_RES (2): Hydrophobic amino acid
  MOD_RES (3): Small amino acid
  MOD_RES (4)..(5): Variable amino acid
  MOD_RES (6): Hydrophobic amino acid
  MOD_RES (7): Variable amino acid
  MOD_RES (8): Variable amino acid and its side chain is linked to position 1 by a linker
  MOD_RES (9): Hydrophobic amino acid
  MOD_RES (10): Small amino acid
  MOD_RES (11): Negatively charged amino acid
  MOD_RES (12): Variable amino acid
  MOD_RES (13): Hydrophobic amino acid
  see specification as filed for detailed description of substitutions and preferred embodiments
XXXXXXXXXXXXX

SEQ ID NO: 6 - protein, 10 aa, Artificial Sequence
  Description of Artificial Sequence Synthetic formula peptide m=0
  MOD_RES (1): Variable amino acid and its side chain is linked to position 8 by a linker
  MOD_RES (2): Variable amino acid
  MOD_RES (3): Hydrophobic amino acid
  MOD_RES (4): Small amino acid
  MOD_RES (5)..(6): Variable amino acid
  MOD_RES (7): Hydrophobic amino acid
  MOD_RES (8): Variable amino acid and its side chain is linked to position 1 by a linker
  MOD_RES (9): Variable amino acid
  MOD_RES (10): Hydrophobic amino acid
  see specification as filed for detailed description of substitutions and preferred embodiments
XXXXXXXXXX

SEQ ID NO: 7 - protein, 14 aa, Artificial Sequence
  Description of Artificial Sequence Synthetic formula peptide m=1
  MOD_RES (1): Variable amino acid and its side chain is linked to position 8 by a linker
  MOD_RES (2): Variable amino acid
  MOD_RES (3): Hydrophobic amino acid
  MOD_RES (4): Small amino acid
  MOD_RES (5)..(6): Variable amino acid
  MOD_RES (7): Hydrophobic amino acid
  MOD_RES (8): Variable amino acid and its side chain is linked to position 1 by a linker
  MOD_RES (9): Variable amino acid
  MOD_RES (10): Hydrophobic amino acid
  MOD_RES (11): Small amino acid
  MOD_RES (12): Negatively charged amino acid
  MOD_RES (13): Variable amino acid
  MOD_RES (14): Hydrophobic amino acid
  see specification as filed for detailed description of substitutions and preferred embodiments
XXXXXXXXXXXXXX

SEQ ID NO: 8 - protein, 9 aa, Artificial Sequence
  Description of Artificial Sequence Synthetic formula peptide p=0
  MOD_RES (1): Variable amino acid and its side chain is linked to position 8 by a linker
  MOD_RES (2): Hydrophobic amino acid
  MOD_RES (3)..(4): Variable amino acid
  MOD_RES (5): Hydrophobic amino acid
  MOD_RES (6): Small amino acid
  MOD_RES (7): Negatively charged amino acid
  MOD_RES (8): Variable amino acid and its side chain is linked to position 1 by a linker
  MOD_RES (9): Hydrophobic amino acid
  see specification as filed for detailed description of substitutions and preferred embodiments
XXXXXXXXX

SEQ ID NO: 9 - protein, 12 aa, Artificial Sequence
  Description of Artificial Sequence Synthetic formula peptide p=1
  MOD_RES (1): Hydrophobic amino acid
  MOD_RES (2): Small amino acid
  MOD_RES (3): Variable amino acid
  MOD_RES (4): Variable amino acid and its side chain is linked to position 11 by a linker
  MOD_RES (5): Hydrophobic amino acid
  MOD_RES (6)..(7): Variable amino acid
  MOD_RES (8): Hydrophobic amino acid
  MOD_RES (9): Small amino acid
  MOD_RES (10): Negatively charged amino acid
  MOD_RES (11): Variable amino acid and its side chain is linked to position 4 by a linker
  MOD_RES (12): Hydrophobic amino acid
  see specification as filed for detailed description of substitutions and preferred embodiments
XXXXXXXXXXXX

SEQ ID NO: 10 - protein, 9 aa, Artificial Sequence
  Description of Artificial Sequence Synthetic formula peptide n=0
  MOD_RES (1): Hydrophobic amino acid
  MOD_RES (2): Variable amino acid and its side chain is linked to position 9 by a linker
  MOD_RES (3): Variable amino acid
  MOD_RES (4): Hydrophobic amino acid
  MOD_RES (5): Small amino acid
  MOD_RES (6): Negatively charged amino acid
  MOD_RES (7): Variable amino acid
  MOD_RES (8): Hydrophobic amino acid
  MOD_RES (9): Variable amino acid and its side chain is linked to position 2 by a linker
  see specification as filed for detailed description of substitutions and preferred embodiments
XXXXXXXXX

SEQ ID NO: 11 - protein, 13 aa, Artificial Sequence
  Description of Artificial Sequence Synthetic formula peptide n=1
  MOD_RES (1): Hydrophobic amino acid
  MOD_RES (2): Small amino acid
  MOD_RES (3)..(4): Variable amino acid
  MOD_RES (5): Hydrophobic amino acid
  MOD_RES (6): Variable amino acid and its side chain is linked to position 13 by a linker
  MOD_RES (7): Variable amino acid
  MOD_RES (8): Hydrophobic amino acid
  MOD_RES (9): Small amino acid
  MOD_RES (10): Negatively charged amino acid
  MOD_RES (11): Variable amino acid
  MOD_RES (12): Hydrophobic amino acid
  MOD_RES (13): Variable amino acid and its side chain is linked to position 6 by a linker
  see specification as filed for detailed description of substitutions and preferred embodiments
XXXXXXXXXXXXX

SEQ ID NO: 12 - protein, 10 aa, Artificial Sequence
  Description of Artificial Sequence Synthetic formula peptide n=0
  MOD_RES (1): Hydrophobic amino acid
  MOD_RES (2): Variable amino acid
  MOD_RES (3): Variable amino acid and its side chain is linked to position 10 by a linker
  MOD_RES (4): Hydrophobic amino acid
  MOD_RES (5): Small amino acid
  MOD_RES (6): Negatively charged amino acid
  MOD_RES (7): Variable amino acid
  MOD_RES (8): Hydrophobic amino acid
  MOD_RES (9): Variable amino acid
  MOD_RES (10): Variable amino acid and its side chain is linked to position 3 by a linker
  see specification as filed for detailed description of substitutions and preferred embodiments
XXXXXXXXXX

SEQ ID NO: 13 - protein, 14 aa, Artificial Sequence
  Description of Artificial Sequence Synthetic formula peptide n=1
  MOD_RES (1): Hydrophobic amino acid
  MOD_RES (2): Small amino acid
  MOD_RES (3)..(4): Variable amino acid
  MOD_RES (5): Hydrophobic amino acid
  MOD_RES (6): Variable amino acid
  MOD_RES (7): Variable amino acid and its side chain is linked to position 14 by a linker
  MOD_RES (8): Hydrophobic amino acid
  MOD_RES (9): Small amino acid
  MOD_RES (10): Negatively charged amino acid
  MOD_RES (11): Variable amino acid
  MOD_RES (12): Hydrophobic amino acid
  MOD_RES (13): Variable amino acid
  MOD_RES (14): Variable amino acid and its side chain is linked to position 7 by a linker
  see specification as filed for detailed description of substitutions and preferred embodiments
XXXXXXXXXXXXXX

SEQ ID NO: 14 - protein, 9 aa, Artificial Sequence
  Description of Artificial Sequence Synthetic peptide
  MOD_RES (1): Variable amino acid and its side chain is linked to position 8 by a linker
  MOD_RES (2): Hydrophobic amino acid
  MOD_RES (3)..(4): Variable amino acid
  MOD_RES (5): Hydrophobic amino acid
  MOD_RES (6): Small amino acid
  MOD_RES (7): Negatively charged amino acid
  MOD_RES (8): Variable amino acid and its side chain is linked to position 1 by a linker
  MOD_RES (9): Hydrophobic amino acid
  see specification as filed for detailed description of substitutions and preferred embodiments
XXXXXXXXX

SEQ ID NO: 15 - protein, 13 aa, Artificial Sequence
  Description of Artificial Sequence Synthetic peptide
  MOD_RES (1): Asp, Lys, Glu or Orn and this residue is linked to position 8 by a linker
  MOD_RES (8): Asp, Lys, Glu or Orn and this residue is linked to position 1 by a linker
  see specification as filed for detailed description of substitutions and preferred embodiments
XIAQELRXIGDEF

SEQ ID NO: 16 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLRRIGDXF

SEQ ID NO: 17 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLRRIGDXF

SEQ ID NO: 18 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAKXLAKIGDXF

SEQ ID NO: 19 - protein, 13 aa, Artificial Sequence
  Description of Artificial Sequence Synthetic peptide
  MOD_RES (5): Asp, Lys, Glu or Orn and this residue is linked to position 12 by a linker
  MOD_RES (12): Asp, Lys, Glu or Orn and this residue is linked to position 5 by a linker
  see specification as filed for detailed description of substitutions and preferred embodiments
KYAKXLAKIGDXF

SEQ ID NO: 20 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
YGRXLRRMSDXF

SEQ ID NO: 21 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IARXLAQVGDXM

SEQ ID NO: 22 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
VGRXLAIIGDXI

SEQ ID NO: 23 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLSSIGSXF

SEQ ID NO: 24 - protein, 12 aa, Artificial Sequence
  Description of Artificial Sequence Synthetic peptide
  MOD_RES (2): Aib
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IXQXLRRIADXF

SEQ ID NO: 25 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
YAQXLRRIGDXF

SEQ ID NO: 26 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
AAQXLRRIGDXF

SEQ ID NO: 27 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXARRIGDXF

SEQ ID NO: 28 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLRRAGDXF

SEQ ID NO: 29 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLRRIGDXA

SEQ ID NO: 30 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLSSIGDXF

SEQ ID NO: 31 - protein, 16 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
IAQXLRRIGDXFNASF

SEQ ID NO: 32 - protein, 12 aa, Artificial Sequence
  Description of Artificial Sequence Synthetic peptide
  MOD_RES (4): Asp, Lys, Glu or Orn and this residue is linked to position 11 by a linker
  MOD_RES (11): Asp, Lys, Glu or Orn and this residue is linked to position 4 by a linker
  see specification as filed for detailed description of substitutions and preferred embodiments
KIAXLRRIGDXF

SEQ ID NO: 33 - protein, 13 aa, Artificial Sequence
  Description of Artificial Sequence Synthetic peptide
  MOD_RES (1): Asp or Glu and this residue is linked to position 8 by a linker
  MOD_RES (8): Asp or Glu and this residue is linked to position 1 by a linker
  see specification as filed for detailed description of substitutions and preferred embodiments
XIAQELRXIGDEF

SEQ ID NO: 34 - protein, 12 aa, Artificial Sequence
  Description of Artificial Sequence Synthetic peptide
  MOD_RES (4): Asp or Glu and this residue is linked to position 11 by a linker
  MOD_RES (11): Asp or Glu and this residue is linked to position 4 by a linker
  see specification as filed for detailed description of substitutions and preferred embodiments
IAQXLRRIGDXF

SEQ ID NO: 35 - protein, 12 aa, Artificial Sequence
  Description of Artificial Sequence Synthetic peptide
  MOD_RES (4): Asp or Glu and this residue is linked to position 11 by a linker
  MOD_RES (11): Asp or Glu and this residue is linked to position 4 by a linker
  see specification as filed for detailed description of substitutions and preferred embodiments
IAQXLRRIGDXF

SEQ ID NO: 36 - protein, 12 aa, Artificial Sequence
  Description of Artificial Sequence Synthetic peptide
  MOD_RES (4): Asp or Glu and this residue is linked to position 11 by a linker
  MOD_RES (11): Asp or Glu and this residue is linked to position 4 by a linker
  see specification as filed for detailed description of substitutions and preferred embodiments
IAQXLRRIGDXF

SEQ ID NO: 37 - protein, 13 aa, Artificial Sequence
  Description of Artificial Sequence Synthetic peptide
IAQELRRIGDEFN

SEQ ID NO: 38 - protein, 26 aa, Artificial Sequence
  Description of Artificial Sequence Synthetic peptide
DLRPEIRIAQELRRIGDEFNETYTRR

SEQ ID NO: 39 - protein, 13 aa, Artificial Sequence
  Description of Artificial Sequence Synthetic peptide
IAQELRRIGDEFN

SEQ ID NO: 40 - protein, 26 aa, Artificial Sequence
  Description of Artificial Sequence Synthetic peptide
DLRPEIRIAQELRRIGDEFNETYTRR

SEQ ID NO: 41 - protein, 12 aa, Artificial Sequence
  Description of Artificial Sequence Synthetic peptide
IAQELRRIGDEF

SEQ ID NO: 42 - protein, 13 aa, Artificial Sequence
  Description of Artificial Sequence Synthetic peptide
QIAQELRRIGDEF

SEQ ID NO: 43 - protein, 13 aa, Artificial Sequence
  Description of Artificial Sequence Synthetic peptide
  MOD_RES (1): This residue is linked to position 8 by a linker
  MOD_RES (8): This residue is linked to position 1 by a linker
  see specification as filed for detailed description of substitutions and preferred embodiments
EIAQELREIGDEF

SEQ ID NO: 44 - protein, 12 aa, Artificial Sequence
  Description of Artificial Sequence Synthetic peptide
  MOD_RES (4): This residue is linked to position 11 by a linker
  MOD_RES (11): This residue is linked to position 4 by a linker
  see specification as filed for detailed description of substitutions and preferred embodiments
IAQELRRIGDEF

SEQ ID NO: 45 - protein, 14 aa, Artificial Sequence
  Description of Artificial Sequence Synthetic peptide
IWIAQELRRIGDEF

SEQ ID NO: 46 - protein, 16 aa, Artificial Sequence
  Description of Artificial Sequence Synthetic peptide
  MOD_RES (2): This residue is linked to position 9 by a linker
  MOD_RES (9): This residue is linked to position 2 by a linker
  see specification as filed for detailed description of substitutions and preferred embodiments
IEIAQELREIGDEFNA

SEQ ID NO: 47 - protein, 16 aa, Artificial Sequence
  Description of Artificial Sequence Synthetic peptide
  MOD_RES (6): This residue is linked to position 13 by a linker
  MOD_RES (13): This residue is linked to position 6 by a linker
  see specification as filed for detailed description of substitutions and preferred embodiments
IWIAQELRRIGDEFNA

SEQ ID NO: 48 - protein, 16 aa, Artificial Sequence
  Description of Artificial Sequence Synthetic peptide
  MOD_RES (9): This residue is linked to position 16 by a linker
  MOD_RES (16): This residue is linked to position 9 by a linker
  see specification as filed for detailed description of substitutions and preferred embodiments
IWIAQELREIGDEFNE

SEQ ID NO: 49 - protein, 13 aa, Artificial Sequence
  Description of Artificial Sequence Synthetic peptide
EIAQELREIGDEF

SEQ ID NO: 50 - protein, 16 aa, Artificial Sequence
  Description of Artificial Sequence Synthetic peptide
IWIAQELRRIGDEFNA

SEQ ID NO: 51 - protein, 16 aa, Artificial Sequence
  Description of Artificial Sequence Synthetic peptide
  MOD_RES (6): This residue is linked to position 13 by a linker
  MOD_RES (13): This residue is linked to position 6 by a linker
  see specification as filed for detailed description of substitutions and preferred embodiments
QAIAQELRRIGDEFNA

SEQ ID NO: 52 - protein, 16 aa, Artificial Sequence
  Description of Artificial Sequence Synthetic peptide
IWIAQQLRRIGDQFNA

SEQ ID NO: 53 - protein, 16 aa, Artificial Sequence
  Description of Artificial Sequence Synthetic peptide
IWAAQELRRIGDEFNA

SEQ ID NO: 54 - protein, 16 aa, Artificial Sequence
  Description of Artificial Sequence Synthetic peptide
IWIAQEARRIGDEFNA

SEQ ID NO: 55 - protein, 16 aa, Artificial Sequence
  Description of Artificial Sequence Synthetic peptide
IWIAQELRRAGDEFNA

SEQ ID NO: 56 - protein, 16 aa, Artificial Sequence
  Description of Artificial Sequence Synthetic peptide
IWIAQELRRIGDEANA

SEQ ID NO: 57 - protein, 16 aa, Artificial Sequence
  Description of Artificial Sequence Synthetic peptide
IWAAQEARRAGDEANA

SEQ ID NO: 58 - protein, 16 aa, Artificial Sequence
  Description of Artificial Sequence Synthetic peptide
IFIAQELRRIGDEFNA

SEQ ID NO: 59 - protein, 16 aa, Artificial Sequence
  Description of Artificial Sequence Synthetic peptide
AWIAQELRRIGDEFNA

SEQ ID NO: 60 - protein, 16 aa, Artificial Sequence
  Description of Artificial Sequence Synthetic peptide
IAIAQELRRIGDEFNA

SEQ ID NO: 61 - protein, 16 aa, Artificial Sequence
  Description of Artificial Sequence Synthetic peptide
IRIAQELRRIGDEFNA

SEQ ID NO: 62 - protein, 16 aa, Artificial Sequence
  Description of Artificial Sequence Synthetic peptide
IWIAQELRRIGDEFAN

SEQ ID NO: 63 - protein, 16 aa, Artificial Sequence
  Description of Artificial Sequence Synthetic peptide
IWIAQELRRIGDEFAA

SEQ ID NO: 64 - protein, 16 aa, Artificial Sequence
  Description of Artificial Sequence Synthetic peptide
  MOD_RES (8)..(9): Cit
IWIAQELXXIGDEFNA

SEQ ID NO: 65 - protein, 16 aa, Artificial Sequence
  Description of Artificial Sequence Synthetic peptide
IWIAQELRRIGDEFNN

SEQ ID NO: 66 - protein, 16 aa, Artificial Sequence
  Description of Artificial Sequence Synthetic peptide
REIGAQLRRMADDLNA

SEQ ID NO: 67 - protein, 16 aa, Artificial Sequence
  Description of Artificial Sequence Synthetic peptide
VQIARKLQAIADQFHR

SEQ ID NO: 68 - protein, 26 aa, Artificial Sequence
  Description of Artificial Sequence Synthetic peptide
DMRPEIWIAQELRRIGDEFNAYYARR

SEQ ID NO: 69 - protein, 16 aa, Artificial Sequence
  Description of Artificial Sequence Synthetic peptide
  MOD_RES (1): D-Ala
  MOD_RES (2): D-Asn
  MOD_RES (3): D-Phe
  MOD_RES (4): D-Glu
  MOD_RES (5): D-Asp
  MOD_RES (6): D-Gly
  MOD_RES (7): D-Ile
  MOD_RES (8)..(9): D-Arg
  MOD_RES (10): D-Leu
  MOD_RES (11): D-Glu
  MOD_RES (12): D-Gln
  MOD_RES (13): D-Ala
  MOD_RES (14): D-Ile
  MOD_RES (15): D-Trp
  MOD_RES (16): D-Ile
ANFEDGIRRLEQAIWI

SEQ ID NO: 70 - protein, 6 aa, Artificial Sequence
  Description of Artificial Sequence Synthetic 6xHis tag
HHHHHH

(19) United States
(12) Patent Application Publication
(10) Pub. No.: US 2007/0032417 A1
(43) Pub. Date: Feb. 8, 2007

(54) Peptides and therapeutic uses thereof

(75) Inventor: Jonathan Baell, Victoria (AU)

Correspondence Address: HELLER EHRMAN WHITE & MCAULIFFE LLP, 1717 RHODE ISLAND AVE, NW, WASHINGTON, DC, 20036-3001, US

(73) Assignee: Walter and Eliza Hall Institute of Medical Research, Parkville (AU)

(21) Appl. No.: 10/540,390

(22) PCT Filed: Dec. 24, 2003
(86) PCT No.: PCT/AU03/01724

§ 371 Date: Jan. 3, 2006

(30) Foreign Application Priority Data
Dec. 24, 2002 (AU) 2002953561

(51) Int. Cl. A61K 38/17; C07K 14/47; C07K 7/08

(52) U.S. Cl. 514/12; 514/13; 530/324; 530/326

(57) ABSTRACT

Conformationally constrained peptides that mimic BH3-only proteins, compositions containing them and their use in the regulation of cell death are disclosed. The conformationally constrained peptides are capable of binding to and neutralising pro-survival Bcl-2 proteins. Processes for preparing the conformationally constrained peptides and their use in the treatment and/or prophylaxis of diseases or conditions associated with deregulation of cell death are also disclosed.

### FIELD OF THE INVENTION

This invention relates generally to conformationally constrained peptides that mimic BH3-only proteins, to compositions containing them and to their use in the regulation of cell death. More particularly the invention relates to conformationally constrained peptides that mimic BH3-only proteins that are capable of binding to and neutralizing pro-survival Bcl-2 proteins. The present invention also relates to processes of preparing the conformationally constrained peptides and to their use in the treatment and/or prophylaxis of diseases or conditions associated with the deregulation of cell death.

### BACKGROUND OF THE INVENTION

Bibliographic details of various publications referred to in this specification are collected at the end of the description.

In the last decade, much has been learnt about the molecular control of programmed cell death (apoptosis), the evolutionary conserved process of killing and removing excess, unwanted or damaged cells during development and in tissue homeostasis. Since the deregulation of apoptosis has been linked to a number of disease states, our understanding of how this process is controlled may allow novel ways to treat diseases, either by promoting or by inhibiting apoptosis (Thompson, 1995). For example, loss of myocardial tissues after acute myocardial infarcts may be limited by inhibiting apoptosis in the damaged tissues. Excessive apoptosis is also a feature of neurodegenerative conditions such as Alzheimer's disease, suggesting that drugs preserving neuronal integrity may have a role in delaying the loss of vital neurons. In contrast to excess cell death, insufficient apoptosis is a feature of malignant disease and autoimmunity (Strasser et al, 1997). In either condition, persistence of damaged or unwanted cells that should normally be removed can contribute to disease.

In malignancies, mutations affecting cell death regulatory proteins or those that sense cellular damage have been described in various tumors. Bcl-2, the prototypic member of the Bcl-2 family of proteins, was first discovered as the result of the t( 11;14) chromosomal translocation in human follicular B-cell lymphoma which results in its overexpression (Tsujimoto et al, 1985; Cleary et al, 1986). Overexpression of Bcl-2, which functions to inhibit apoptosis (Vaux et al, 1988) or its functional homologs have also been reported in other tumors. However, mutations to proteins involved in sensing DNA damage are much more common, in tumors. It is estimated that over half of human cancers have a mutation of the tumor suppressor protein, p53, or ones affecting this pathway (Lane, 1992). p53 is necessary to elicit the appropriate cellular responses (growth arrest, apoptosis) to most forms of DNA damage. Interestingly p53 kills cells mainly by a Bcl-2-dependent mechanism since Bcl-2 overexpression can block most cell deaths induced by p53 (Lowe et al., 1993; Strasser et al., 1994). Both clinical observations and experiments in mouse models suggest that inhibition of apoptosis (e.g. p53 mutations, overexpression of Bcl-2) (Strasser et al, 1990; Adams et al, 1992) greatly promote oncogenic transformation caused by mutations that promote cellular proliferation alone (e.g. overexpression of c-Myc, p21^{ras} mutations). Thus, reversing the process of tumorigenesis by promoting cell death, such as by activating p53 function or by inhibiting Bcl-2 function, may allow novel ways to complement our current treatments for malignancies. Furthermore, most of the cytotoxic treatments currently used to treat malignant diseases work partly by inducing the endogenous cell death machinery (Fisher, 1994), although this has been disputed by others (Brown and Wouters, 1999). For example, radiotherapy and many chemotherapeutic drugs activate apoptotic machinery indirectly by inducing DNA damage. Since the majority of tumors have mutations affecting the p53 pathway, forms of therapy that target the p53 pathway are significantly blunted because of the frequent loss of p53 function. The resistance of tumor cells to conventional agents provides further impetus to discovering novel cytotoxic drugs that act directly on the cell death machinery.

The effectors of cell death are cysteine proteases of the caspase family that cleave vital cellular substrates after aspartate residues (Thomberry, 1998). The caspases are synthesised as inactive zymogens and are only activated in response to cellular damage, thereby allowing exquisite control of apoptosis during normal tissue functioning in order to prevent inappropriate cell deaths. There are at least two distinct pathways to activate caspases in mammalian cells (Strasser et al, 2000). Binding of cognate ligands to some members of the TNF receptor superfamily induce cell death by activating the initiator caspase, caspase-8/FLICE, which is recruited to form oligomers on the receptor by the adaptor protein FADD/MORT-1 (Ashkenazi and Dixit, 1998). Once activated, caspase-8 can cleave downstream effector caspases such as caspases-3, -6, and -7, thereby massively amplifying the process.

A second pathway to caspase activation is that controlled by the Bcl-2 family of proteins (Adams and Cory, 2001). Overexpression of Bcl-2 can block many forms of physiologically (e.g., developmentally programmed cell deaths, death due to growth factor deprivation) and experimentally applied damage signals (e.g., cellular stress, radiation, most chemotherapeutic drugs). Bcl-2 controls the activation of the initiator caspase, caspase-9, by the adaptor protein Apaf-1, but this does not appear to be the critical or the sole molecule regulated by Bcl-2 (Moriishi et al, 1999; Conus et al, 2000; Hausmann et al, 2000; Haraguchi et al, 2000; Marsden et al., 2002). In the nematode C. elegans, the Bcl-2 homologue CED-9 functions by sequestering the activity of the adaptor protein CED-4 which is required to activate the caspase CED-3 (Spector et al, 1997; Chinnaiyan et al, 1997; Wu et al, 1997; Yang et al, 1998; Chen et al, 2000). However, a true mammalian homologue of CED-4 has not been discovered to date. The machinery is also more complex in mammals which express a number of structural and functional homologues of Bcl-2, namely Bcl-x_{L}, Bcl-w, Mcl-1 and A1 (Adams and Cory, 1998) (Cory and Adams, 2002). These pro-survival proteins are structurally similar, generally containing four conserved Bcl-2 homology domains (BH1-4), as well as a C-terminal hydrophobic region, promoting cell survival until antagonised by a family of distantly related proteins, the BH3-only proteins (Baell J and Huang D C, 2002).

The BH3-only proteins are so-called because they share with each other, and with the other members of the Bcl-2 family of proteins, only the short BH3 domain (Huang and Strasser, 2000). This short domain forms an α-helical region, the hydrophobic face of which binds onto a hydrophobic surface cleft present on pro-survival Bcl-2 (Sattler et al, 1997; Petros et al, 2000). The BH3-only proteins probably function to sense cellular damage to critical cellular structures or metabolic process, and are then unleashed to initiate cell death by binding to and neutralising Bcl-2 (Huang and Strasser, 2000; Bouillet et al, 1999). Normally, the BH3-only proteins are kept inert by transcriptional or translational mechanisms, thereby preventing inappropriate cell deaths. Recently, two BH3-only proteins that are transcriptional targets of the tumour suppressor protein p53 have been described, namely Noxa (Oda et al, 2000) and Puma/Bbc3 (Yu et al, 2001; Nakano and Wousden, 2001; Han et al, 2001). These proteins are thus good candidates to mediate cell death induced by p53 activation (Vousden, 2000). Some other BH3-only proteins are controlled instead by post-translational mechanisms. In particular, two are sequestered to the cell's cytoskeletal network, Bim to the microtubules and Bmf to the actin cytoskeleton (Puthalakath et al, 1999; Puthalakath et al, 2001). Damage signals that impinge upon these cytoskeletal structures will activate Bim or Bmf freeing them to bind to pro-survival Bcl-2 located on the cytoplasmic face of the outer mitochondrial membrane as well as those of the nucleus and endoplasmic reticulum.

Recently it has been shown that the killing by the BH3-only proteins is dependent on the activity of a third family of Bcl-2-related proteins, namely the Bax sub-family (Zong et al., 2001; Cheng et al., 2001). Although these proteins bear three of the four homology domains and are structurally very similar to the pro-survival proteins (Suzuki et al, 2001), Bax, Bak and Bok/Mtd function instead to promote cell death. Biochemically, damage signals cause these proteins to aggregate and such oligomers may function to cause damage to mitochondrial membranes (Eskes et al., 2000; Desagher et al, 1999; Antonsson et al; 2001; Mikhailov et al., 2001; Wei et al., 2001; Juirgensmeier et al., 1998), thereby causing the release of mitochondrial pro-apoptogenic factors such as Smac/Diablo (Verhagen et al., 2000; Du et al., 2000) and cytochrome c, which is essential for the activation of caspase-9 by Apaf-1 (Kluck et al., 1997; Yang et al., 1997; Zou et al., 1997; Li et al., 1997). Since killing by BH3-only proteins depends on Bax and Bak in fibroblasts, their physiological role may be to activate Bax and Bak (Zong et al., 2001; Korsmeyer et al., 2000). In such a model, the pro-survival Bcl-2 proteins function merely to sequester the BH3-only proteins until such time as when there is insufficient capacity to do so. However, there are few reports of direct binding of the BH3-only proteins to Bax and Bak and even in the case of the BH3-only protein Bid appears weak (Eskes et al., 2000; Wei et al., 2001; Wang et al., 1996). To date there are no experiments to directly compare the binding of BH3-only proteins with pro-survival Bcl-2 and to pro-apoptotic Bax.

In addition to the tenuous biochemical evidence for the direct binding of BH3-only proteins to Bax-like proteins, careful analyses of the available genetic data also suggests that pro-survival Bcl-2 rather than pro-apoptotic Bax is the direct target of BH3-only proteins. In the nematode C. elegans, all the killing induced by the BH3-only protein EGL-1 is dependent on the ability of EGL-1 to bind to and neutralise nematode Bcl-2, CED-9 (Conradt et al., 1998; Parrish et al., 2000). The situation is somewhat more complex in mammals because of the functional redundancy in each class of proteins. Instead of a single BH3-only protein (EGL-1) and a single Bcl-2 homologue (CED-9), mammals express multiple proteins of each sub-class making direct comparison with the nematode difficult. Furthermore, nematodes do not appear to express Bax-like proteins. However, if the Bcl-2-like proteins function merely to sequester BH3-only proteins, then the amount of pro-survival Bcl-2-like proteins in any cell type must be limiting. It is therefore surprising that mice lacking a single allele of the bcl-2 (Veis et al., 1993; Nakayama et al., 1994; Kamada et al., 1995), bcl-x (Motoyama et al., 1995; Motoyama et al., 1999) or bcl-w (Ross et al., 1998; Print et al., 1998) genes are normal whereas the homozygous knock-out mice all have striking phenotypes in the cell types where these genes play a crucial role. This suggests that the pro-survival Bcl-2-like proteins are not limiting; instead analysis of mice lacking the BH3-only protein Bim suggest that this class of proteins is limiting (Bouillet et al., 1999; Bouillet et al., 2001). Taken together, the available data would suggest that BH3-only proteins directly bind to Bcl-2 and it is by neutralising Bcl-2 that BH3-only proteins can activate Bax-like proteins.

Thus, agents that directly mimic the BH3-only proteins may induce cell death and may therefore be of value therapeutically. As Bcl-2 controls the critical point that determines a cell's fate, this class of proteins represent an attractive target for drug design. In particular, since many of the oncogenic mutations, such as those to p53, result in defects in sensing cellular damage that would normally result in cell death by a Bcl-2-dependent mechanism, directly targeting Bcl-2 and its homologs may circumvent such mutations. This may also permit an alternative route to overcome tumor resistance to current treatments.

### SUMMARY OF THE INVENTION

The present invention is predicated in part on the discovery that conformationally constrained peptides that mimic BH3-only proteins exhibit significant pro-apoptotic activity and have increased resistance to proteolysis compared to unconstrained linear peptides. This discovery has been reduced to practice in novel compounds, in compositions containing them and in methods for their preparation and use, as described hereinafter.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers of steps.

In a first aspect of the invention there is provided a conformationally constrained compound or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker (L) which tethers two amino acid residues, Zaa₁ and Zaa₂, in the sequence.

As used herein, the term “conformationally constrained” refers the stabilization of a desired conformation, preferably a helical conformation, relative to other possible conformations by means of a linker which is covalently bound to two amino acid residues in the sequence. The conformational constraint also increases resistance to proteolysis compared to peptides lacking conformational constraint.

As used herein, the term “amino acid” refers to compounds having an amino group and a carboxylic acid group. An amino acid may be a naturally occurring amino acid or non-naturally occurring amino acid and may be a proteogenic amino acid or a non-proteogenic amino acid. The amino acids incorporated into the amino acid sequences of the present invention may be L-amino acids, D-amino acids, α-amino acid, β-amino acids, sugar amino acids and/or mixtures thereof.

Suitable naturally occurring proteogenic amino acids are shown in Table 1 together with their one letter and three letter codes.

TABLE 1

| Amino Acid | one letter code | three letter code |
|---|---|---|
| L-alanine | A | Ala |
| L-arginine | R | Arg |
| L-asparagine | N | Asn |
| L-aspartic acid | D | Asp |
| L-cysteine | C | Cys |
| L-glutamine | Q | Gln |
| L-glutamic acid | E | Glu |
| glycine | G | Gly |
| L-histidine | H | His |
| L-isoleucine. | I | Ile |
| L-leucine | L | Leu |
| L-lysine | K | Lys |
| L-methionine | M | Met |
| L-phenylalanine | F | Phe |
| L-proline | P | Pro |
| L-serine | S | Ser |
| L-threonine | T | Thr |
| L-tryptophan | W | Trp |
| L-tyrosine | Y | Tyr |
| L-valine | V | Val |

Suitable non-proteogenic or non-naturally occurring amino acids may be prepared by side chain modification or by total synthesis. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via Q-acylisourea formation followed by subsequent derivitisation, for example, to a corresponding amide.

Sulfydryl groups may be modified by methods such as carboxymethylation with iodoacetic acid or iodoacetamide; performic acid oxidation to cysteic acid; formation of a mixed disulfides with other thiol compounds; reaction with maleimide, maleic anhydride or other substituted maleimide; formation of mercurial derivatives using 4-chloromercuribenzoate, 4-chloromercuriphenylsulfonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulfenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Examples of suitable non-proteogenic or non-naturally occurring amino acids contemplated herein is shown in Table 2.

TABLE 2

| | | Non-conventional | |
|---|---|---|---|
| Non-conventional amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl | Nmbc | | |
| ethylamino)cyclopropane | | | |

Suitable β-amino acids include, but are not limited to, L-β-homoalanine, L-β-homoarginine, L-β-homoasparagine, L-β-homoaspartic acid, L-β-homoglutamic acid, L-β-homoglutamine, L-β-homoisoleucine, L-β-homoleucine, L-β-homolysine, L-β-homomethionine, L-β-homophenylalanine, L-β-homoproline, L-β-homoserine, L-β-homothreonine, L-β-homotryptophan, L-β-homotyrosine, L-β-homovaline, 3-amino-phenylpropionic acid, 3-amino-chlorophenylbutyric acid, 3-amino-fluorophenylbutyric acid, 3-amino-bromopheynyl butyric acid, 3-amino-nitrophenylbutyric acid, 3-amino-methylphenylbutyric acid, 3-amino-pentanoic acid, 2-amino-tetrahydroisoquinoline acetic acid, 3-amino-naphthyl-butyric acid, 3-amino-pentafluorophenyl-butyric acid, 3-amino-benzothienyl-butyric acid, 3-amino-dichlorophenyl-butyric acid, 3-amino-difluorophenyl-butyric acid, 3-amino-iodophenyl-butyric acid, 3-amino-trifluoromethylphenyl-butyric acid, 3-amino-cyanophenyl-butyric acid, 3-amino-thienyl-butyric acid, 3-amino-5-hexanoic acid, 3-amino-furyl-butyric acid, 3-amino-diphenyl-butyric acid, 3-amino-6-phenyl-5-hexanoic acid and 3-amino-hexynoic acid.

Sugar amino acids are sugar moieties containing at least one amino group as well as at least one carboxyl group. Sugar amino acids may be based on pyranose sugars or furanose sugars. Suitable sugar amino acids may have the amino and carboxylic acid groups attached to the same carbon atom, cc-sugar amino acids, or attached to adjacent carbon atoms, β-sugar amino acids. Suitable sugar amino acids include but are not limited to

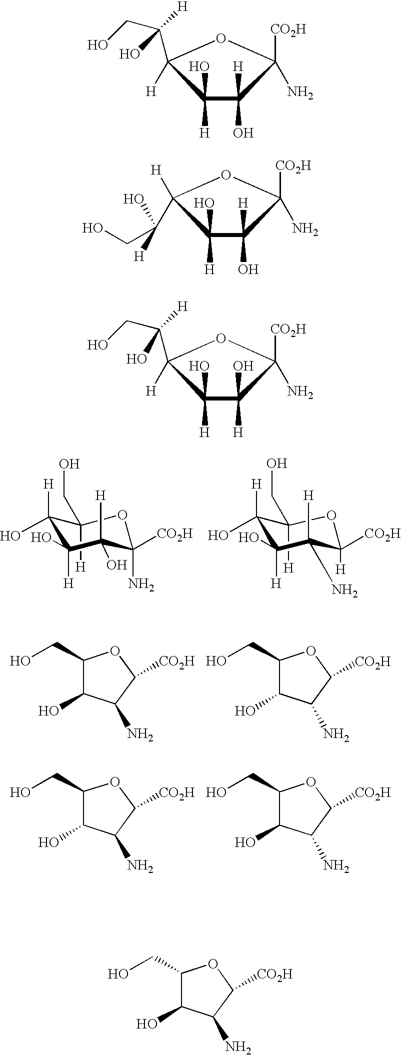

Sugar amino acids may be synthesized starting from commercially available monosaccharides, for example, glucose, glucosamine and galactose. The amino group may be introduced as an azide, cyanide or nitromethane group with subsequent reduction. The carboxylic acid group may be introduced directly as CO₂, by Wittig reaction with subsequent oxidation or by selective oxidation of a primary alcohol.

Haa₁, Haa₂, Haa₃ and Haa₄ are amino acids having hydrophobic side chains and provide the hydrophobic moieties for binding with the Bcl-2 protein. Haa₃ and at least two of Haa₁, Haa₂, and Haa₄ are required for binding. When one of Haa₁, Haa₂, and Haa₄ are not an amino acid having a hydrophobic side chain, they may be any amino acid as described for Xaa₁ below. Preferably all of Haa₁, Haa₂, Haa₃ and Haa₄ are amino acids having a hydrophobic side chain. Suitable Haa₁, Haa₂, Haa₃ and Haa₄ are selected from L-phenylalanine, L-isoleucine, L-leucine, L-valine, L-methionine, L-tyrosine, D-phenylalanine, D-isoleucine, D-leucine, D-valine, D-methionine, D-tyrosine, L-β-homophenylalanine, L-β-homoisoleucine, L-β-homoleucine, L-β-homovaline, L-β-homomethionine, L-β-homotyrosine, aminonorbornylcarboxylate, cyclohexylalanine, L-norleucine, L-norvaline, L-α-methyl isoleucine, L-α-methylleucine, L-α-methylmethionine, L-α-methylnorvaline, L-α-methylphenylalanine, L-α-methylvaline, L-α-methyltyrosine, L-α-methylhomophenylalanine, D-α-methylleucine, D-α-methylmethionine, D-α-methylnorvaline, D-α-methylphenylalanine, D-α-methylvaline, D-α-methyltyrosine, D-α-methylhomophenylalanine residues L-tryptophan, L-3′4′-dichlorophenylalanine, L-1′naphthylalanine and L-2′naphthylalanine. Preferably Haa₁, Haa₂, Haa₃ and Haa₄ are independently selected from L-phenylalanine, L-isoleucine, L-leucine, L-valine, L-methionine and L-tyrosine. In a particularly preferred embodiment Haa₂ is L-leucine.

Saa is an amino acid residue having a small side chain. Suitable Saa residues include glycine, L-alanine, L-serine, L-cysteine, D-alanine, D-serine, D-cysteine, L-β-homoserine, L-β-homoalanine, γ-aminobutyric acid, aminoisobutyric acid, L-α-methylserine, L-α-methylalanine L-α-methylcysteine, D-α-methylserine, D-α-methylalanine and D-α-methylcystine residues. Preferably Saa is selected from the group consisting of glycine, L-alanine, L-serine, L-cysteine and aminoisobutyric acid.

Naa is a negatively charged amino acid residue. Suitable Naa residues include L-aspartic acid, L-glutamic acid, D-aspartic acid, D-glutamic acid, L-β-homoaspartic acid, L-β-homoglutamic acid, L-α-methylaspartic acid, L-α-methylglutamic acid, D-α-methylaspartic acid and D-α-methylglutamic acid. Preferably Naa is an L-aspartic acid residue or an L-glutamic acid.

Xaa₁, Xaa₂, Xaa₃, Xaa₄ and Xaa₅ are independently selected from any amino acid as defined above and may be any naturally occurring, non naturally occurring, proteogenic or non-proteogenic amino acid. Preferably Xaa₁, Xaa₂, Xaa₃, Xaa₄ and Xaa₅ are independently selected from L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine and L-valine.

R is selected from H, an N-terminal capping group or an oligopeptide optionally capped by an N-terminal capping group. Preferably R is an N-terminal capping group or an oligopeptide having 1 to 10 amino acid residues selected from Xaa₁, optionally capped by an N-terminal capping group. Preferably the N-terminal capping group is a group that stabilises the terminus of a helix, usually having hydrogen atoms able to form hydrogen bonds or having a negative charge at the N-terminus to match with the helix dipole. Suitable N-terminal capping groups include acyl and N-succinate (HO₂CCH₂C(═O)) (Andrews and Tabor).

R′ is selected from H, a C-terminal capping group or an oligopeptide optionally capped by a C-terminal capping group. Preferably R′ is a C-terminal capping group or an oligopeptide having 1 to 10 amino acids selected from Xaa₁, optionally capped by a C-terminal capping group. Preferably the C-terminal capping group is a group that stabilises the terminus of a helix, usually having hydrogen atoms able to form hydrogen bonds or having a negative charge at the C-terminus to match with the helix dipole. A preferred C-terminal capping group is NH₂.

The linker tethers two amino acid residues in the amino acid sequence. Preferably the linker tethers two non-adjacent amino acids that are suitably in an i(i+7) relationship where a first end of the linker is attached to a first amino acid residue (Zaa₁) at a first position in the sequence and the other end of the linker is attached to a second amino acid residue (Zaa₂) which appears in the sequence 7 amino acids after the first amino acid. Preferably the linker stabilizes a desired conformation, preferably a helical conformation. Preferably the linker has a length of 4 to 8 atoms and Zaa₁ and Zaa₂ are located in the amino acid sequence (I) in one of the following positions:
(a) i before Haa₁ at the N-terminal end of the amino acid sequence and i+7 between Haa₂ and Haa₃;
(b) i between Haa₁ and Haa₂ and i+7 between Haa₃ and Haa₄;
(c) i between Haa₂ and Haa3 and i+7 after Haa₄ at the C-terminal end of the amino acid sequence.

In a preferred embodiment, the linker (L) is 4 to 8 atoms in length. The linker may be a hydrocarbon chain of 4 to 8 carbon atoms in length or one or more of the carbon atoms in the hydrocarbon chain may be replaced by a heteroatom selected from N, O or S. One or more of the atoms in the linker may be substituted with a substituent selected from ═O, OH, SH and CH₃. Alternatively, some of the carbon atoms may be replaced by a 1,4-disubstituted phenyl ring.

Zaa₁ and Zaa₂ may be any amino acid residue, however it is preferred that Zaa₁ and Zaa₂ are amino acid residues having side chains which are easily reacted with the linker precursor to form the linker. In a preferred embodiment, the linker covalently links two amino acid residues by the formation of amide bonds, that is, by forming a lactam bridge. Preferably, Zaa₁ and Zaa₂ are independently selected from L-aspartic acid, L-glutamic acid, L-lysine, L-ornithine, D-aspartic acid, D-glutamic acid, D-lysine, D-ornithine, L-β-homoaspartic acid, L-β-homoglutamic acid, L-β-homolysine, L-α-methylaspartic acid, L-α-methylglutamic acid, L-α-methyllysine, L-α-methylornithine, D-α-methylaspartic acid, D-α-methylglutamic acid, D-α-methyllysine and L-α-methylornithine. Preferably, Zaa₁ and Zaa₂ are selected from L-aspartic acid, L-glutamic acid, L-lysine and L-ornithine. More preferably, Zaa₁ and Zaa₂ are selected from L-aspartic acid and L-glutamic acid.

When Zaa₁ and Zaa₂ have side chains containing a carboxylic acid, for example, L-aspartic acid or L-glutamic acid, preferred linkers are selected from the group consisting of —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)₂N⁺H₂(CH₂)₂NH—, —NH(CH₂)₂S(CH₂)₂NH—, —NHCH₂C(═O)NH(CH₂)₂NH—, —NH(CH₂)₂NHC(═O)CH₂NH—, —NH(CH₂)₂SS(CH₂)₂—NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—. More preferably the linker is selected from the group consisting of —NH(CH₂)₅NH—, —NH(CH₂)₆NH—, —NH(CH₂)₇NH—, —NHCH₂C(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—. Especially preferred linkers include —NH(CH₂)₅NH— and —NHCH₂C(═O)NH(CH₂)₂NH—.

When Zaa₁ and Zaa₂ have side chains containing an amino group, for example, L-lysine or L-ornithine, preferred linkers are selected from the group consisting of —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂—C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH2)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—. More preferably the linker is selected from the group consisting of —C(═O)(CH₂)₅C(═O)—, —C(═O)(CH₂)₆C(═O)—, —C(═O)(CH₂)₇C(═O)—, —C(═O)CH₂C(═O)NH(CH₂)₂C(═O)—, —C(═O)(CH₂)₂NHC(═O)CH₂C(═O)—, —C(═O)(CH₂)₂O(CH₂)₃C(═O)— and —C(═O)(CH₂)₂C(═O)NH(CH₂)₂C(═O)—. Especially preferred linkers include —C(═O)(CH₂)₅C(═O)— and —C(═O)CH₂C(═O)NH(CH₂)₂C(═O)—.

When Zaa₁ has a side chain containing an amino group, for example, L-lysine or L-ornithine, and Zaa₂ has a side chain containing a carboxylic acid group, for example, L-aspartic acid or L-glutamic acid, preferred linkers are selected from the group consisting of —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂—NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(═O)(CH₂)₂C(═O)NH(CH₂)₃NH—, —C(═O)(CH2)₃NHC(═O)(CH₂)₂NH—, —C(═O)(CH₂)₃C(═O)NH(CH₂)₂NH— and —C(═O)(CH₂)₂NHC(═O)(CH₂)₃NH—. More preferably the linker is selected from the group consisting of —C(═O)(CH₂)₅NH—, —C(═O)(CH₂)₆NH—, —C(═O)(CH₂)₇NH—, —C(═O)CH₂C(═O)NH(CH₂)₂NH—, —C(═O)(CH₂)₂NHC(═O)CH₂NH—, —C(═O)(CH₂)₂O(CH₂)₃NH— and —C(═O)(CH₂)₂C(═O)NH(CH₂)₂NH—. Especially preferred linkers include —C(═O)(CH₂)₅NH— and —C(═O)CH₂C(═O)NH(CH₂)₂NH—.

When Zaa₁ has a side chain containing a carboxylic acid group, for example, L-aspartic acid or L-glutamic acid, and Zaa₂ has a side chain containing an amino group, for example, L-lysine or L-ornithine, preferred linkers are selected from the group consisting of —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)—, —NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)— and —NH(CH₂)₂NHC(═O)(CH₂)₃C(═O)—. More preferably the linker is selected from the group consisting of —NH(CH₂)₅C(═O)—, —NH(CH₂)₆C(═O)—, —NH(CH₂)₇C(═O)—, —NHCH₂C(═O)NH(CH₂)₂C(═O)—, —NH(CH₂)₂NHC(═O)CH₂C(═O)—, —NH(CH₂)₂O(CH₂)₃C(═O)— and —NH(CH₂)₂C(═O)NH(CH₂)₂C(═O)—. Especially preferred linkers include —NH(CH₂)₅C(═O)— and —NHCH₂C(═O)NH(CH₂)₂C(═O)—.

Preferably the amino acid sequence of the compound is between 9 and 32 amino acid residues in length, more preferably between 9 and 31 amino acids in length, even more preferably between 9 and 30 amino acids in length, even more preferably between 9 and 29 amino acids in length, even more preferably between 9 and 28 amino acids in length, even more preferably between 9 and 27 amino acids in length, even more preferably between 9 and 26 amino acids in length, even more preferably between 9 and 25 amino acids in length, even more preferably between 9 and 24 amino acids in length, even more preferably between 9 and 23 amino acids in length, even more preferably between 9 and 22 amino acids in length, even more preferably between 9 and 21 amino acid residues in length, even more preferably between 9 and 20 amino acids in length, even more preferably between 9 and 19 amino acids in length, even more preferably between 9 and 18 amino acids in length, even more preferably between 9 and 17 amino acids in length, even more preferably 9 and 16 amino acid residues in length, even more preferably between 9 and 15 amino acids in length, even more preferably between 9 and 14 amino acids in length, and still even more preferably between 9 and 13 amino acids in length. An especially preferred amino acid sequence is between 9 and 12 amino acid residues in length.

Especially preferred conformationally constrained compounds of the invention are as depicted in one of formulae (II) to (VI):

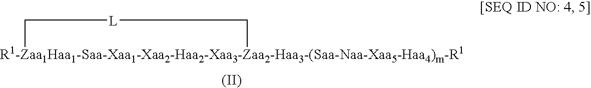

wherein Haa₁, Haa₂, Haa₃, Haa₄, Xaa₁, Xaa₂, Xaa₃, Xaa₅, Saa, Naa and L are as defined above for formula (I), m is 0 or 1, R¹ and R^{1′} are as defined above for R and R′ in formula (I), Zaa₁-L-Zaa₂ represents two amino acid residues with their side chains bridged by a linker L;

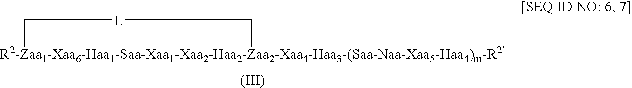

wherein Haa₁, Haa₂, Haa₃, Haa₄, Xaa₁, Xaa₂, Xaa₄, Xaa₅, Saa, Naa and L are as defined above for formula (I), Xaa₆ is an amino acid residue as defined for Xaa₁ above; m is 0 or 1, R² and R^{2′} are as defined above for R and R′ in formula (I), Zaa₁-L-Zaa₂ represents two amino acid residues with their side chains bridged by a linker L;

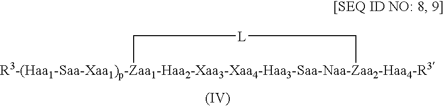

wherein Haa₁, Haa₂, Haa₃, Haa₄, Xaa₁, Xaa₃, Xaa₄, Saa, Naa and L are as defined above for formula (I), p is 0 or 1, R³ and R^{3′} are as defined above for R and R′ in formula (I), Zaa₁-L-Zaa₂ represents two amino acid residues with their side chains bridged by a linker L;

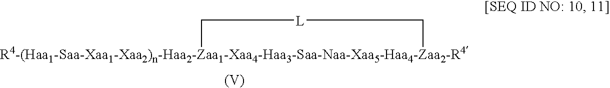

wherein Haa₁, Haa₂, Haa₃, Haa₄, Xaa₁, Xaa₂, Xaa₄, Xaa₅, Saa, Naa and L are as defined above in formula (I), n is 0 or 1, R⁴ and R^{4′} are as defined above for R and R′ in formula (I), Zaa₁-L-Zaa₂ represents two amino acid residues with their side chains bridged by a linker L; and

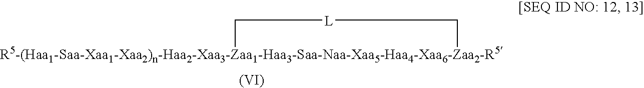

wherein Haa₁, Haa₂, Haa₃, Haa₄, Xaa₁, Xaa₂, Xaa₃, Xaa₅, Saa, Naa and L are as defined above for formula (I), Xaa₆ is an amino acid residue as defined for Xaa₁ above; n is 0 or 1, R⁵ and R^{5′} are as defined above for R and R′ in formula (I), Zaa₁-L-Zaa₂ represents two amino acid residues with their side chains bridged by a linker L; or a pharmaceutically acceptable salt or prodrug thereof.

Especially preferred compounds of the invention include compounds of formula (VII): 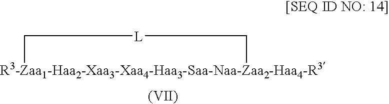
wherein Zaa₁, Haa₂, Xaa₃, Xaa₄, Haa₃, Saa, Naa, Zaa₂, Haa₄, R³, R^{3′} and L are defined above in formula (IV).

Especially preferred compounds of the invention include compounds of formula (VIII): 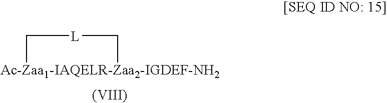
where Zaa₁ and Zaa₂ are selected from L-aspartic acid, L-glutamic acid; and

L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH— and —NH(CH₂)₂NHC(═O)(CH₂)₂NH—; or where Zaa₁ and Zaa₂ are selected from L-lysine and ornithine; and

L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)— and —C(═O)(CH₂)₂NHC(═O)(CH₂)₂C(═O)—; or

where Zaa₁ is selected from L-aspartic acid, L-glutamic acid and Zaa₂ is selected from L-lysine and ornithine; and

L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O (CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)— and —NH(CH₂)₂NHC(═O)(CH₂)₂C(═O)—; or

where Zaa₁ is selected from L-lysine and ornithine and Zaa₂ is selected from L-aspartic acid, L-glutamic acid; and

L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH— and —C(═O)(CH₂)₂NHC(═O)(CH₂)₂NH—;

or compounds of formula (IX)

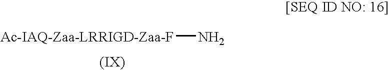

where Zaa₁ and Zaa₂ are selected from L-aspartic acid, L-glutamic acid; and

L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—; or

where Zaa₁ and Zaa₂ are selected from L-lysine and ornithine; and

L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH₂)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—; or

where Zaa₁ is selected from L-aspartic acid, L-glutamic acid and Zaa₂ is selected from L-lysine and ornithine; and

L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)—NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)— and —NH(CH₂)₂NHC(═O)(CH₂)₃C(═O)—; or

where Zaa₁ is selected from L-lysine and ornithine and Zaa₂ is selected from L-aspartic acid, L-glutamic acid; and

L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(═O)(CH₂)₂C(═O)NH(CH₂)₃NH—, —C(═O)(CH₂)₃NHC(═O)(CH₂)₂NH—, —C(═O)(CH₂)₃C(═O)NH(CH₂)₂NH— and —C(═O)(CH₂)₂NHC(═O)(CH₂)₃NH—.

Examples of especially preferred compounds of the invention include: 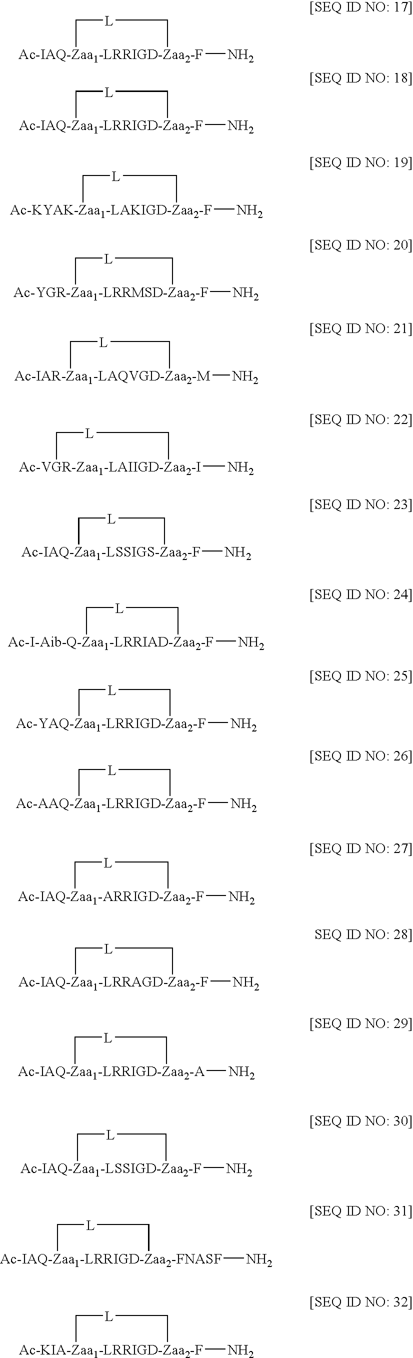
where Zaa₁, Zaa₂ and L are as defined above. Preferably Zaa₁ and Zaa₂ are independently selected from L-aspartic acid and L-glutamic acid and preferably L is selected from —NH(CH₂)₅NH—, —NH(CH₂)₆NH—, —NH(CH₂)₇NH—, —NHCH₂(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—.

Especially preferred linkers include —NH(CH₂)₅NH— and —NHCH₂C(═O)NH(CH₂)₂NH—.

Especially preferred compounds of the invention include: 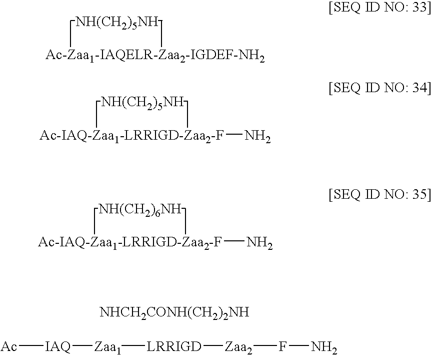
where Zaa₁ and Zaa₂ are independently selected from L-aspartic acid and L-glutamic acid, especially L-glutamic acid.

The compounds of the present invention may be prepared using techniques known in the art. For example, peptides can be synthesized using various solid phase techniques (See Roberge et al.; 1995) or using an automated synthesis, for example, using a Pioneer peptide synthesizer and standard F-moc chemistry, Fields (1991).

The linear peptides can also be prepared using recombinant DNA techniques known in the art. For example, nucleotide sequences encoding a peptide having the required amino acid sequence, can be inserted into a suitable DNA vector, such as a plasmid. Techniques suitable for preparing a DNA vector are described in Sambrook, J., et al., 1989. Once inserted, the vector is used to transform a suitable host. The recombinant peptide is then produced in the host by expression. The transformed host can be either a prokaryotic or eukaryotic cell.

Once the peptides of the present invention have been prepared, they may be substantially purified by preparative HPLC. The composition of the synthetic peptides can be confirmed by amino acid analysis or by sequencing (using the Edman degradation procedure).

Alternatively, a nucleotide sequence encoding amino acid residues 88 to 99 of the Bim protein (relative to the full length Bim protein) can be mutagenised, for example, treated with a chemical mutagen, such as a base analog, a deaminating agent, or an alkylating agent, or with a physical mutagen, such as UV or ionizing radiation or heat, using techniques known in the art. The mutant nucleotide sequence can then be expressed in a suitable host and the recombinant polypeptide purified using standard protocols known to a person skilled in the art.

The linker may be incorporated into the peptide using known techniques. For example, when Zaa₁ and Zaa₂ are residues having an acidic side chain, such as aspartic acid or glutamic acid, each of Zaa₁ and Zaa₂ is selectively protected before the peptide is synthesised. After peptide synthesis, one of the protecting groups (P₁) is selectively removed and the resulting carboxylic acid group is reacted with the amine of the linker to form an amide bond. The other protecting group (P₂) is selectively removed and the second carboxylic acid is reacted with another amine on the linker to form a second amide bond. This process is shown in Scheme 1.

Similarly, when Zaa₁ and Zaa₂ are residues having an amino side chain, such as lysine or ornithine, these residues may be reacted with a dicarboxylic acid. During the reaction, one of the carboxylic acid groups on the dicarboxylic acid linker precursor is selectively protected. The remaining carboxylic acid is reacted with the amine of the lysine or ornithine residue to form an amide bond. The protecting group (P) is removed and the second carboxylic acid is reacted with a second amine on a lysine or ornithine residue to form a second amide bond. This process is shown in Scheme 2.

Suitable protecting and deprotecting methods for reactive functional groups such as carboxylic acids and amines are known in the art, for example, in Protective Groups in Organic Synthesis, T. W. Green & P. Wutz, John Wiley & Son, 3^{rd} Ed, 1999.

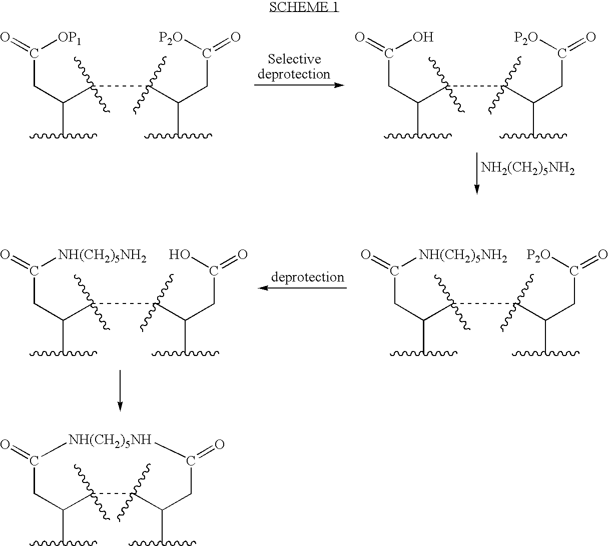

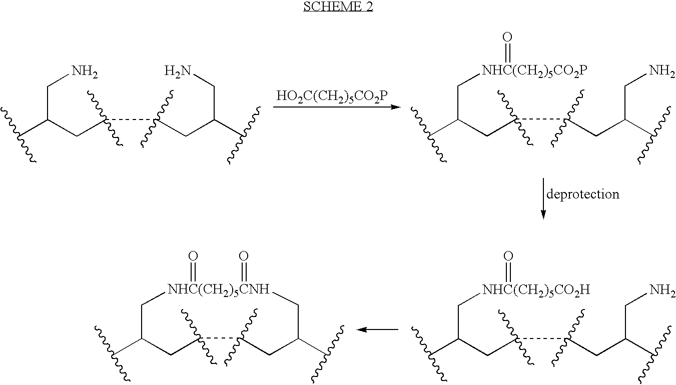

In another aspect of the invention there is provided a screening assay for identifying a candidate compound capable of inducing apoptosis or cell death in cells. The assay is based on the ability of candidate compounds to disrupt, or compete with, the binding of a Bim BH3-peptide comprising the sequence IAQELRRIGDEFN [SEQ ID NO: 37] to a Bcl-2 family protein. The BH3 peptide is preferably labelled. Preferably the Bim BH3 peptide has the sequence:

| | |
|---|---|
| DLRPEIRIAQELRRIGDEFNETYTRR | [SEQ ID NO:38] |

Accordingly, in another aspect of the invention there is provided an assay for identifying compounds which bind to a member of the Bcl-2 family of proteins, the assay comprising the steps of:
(a) providing a candidate compound to be tested;

(b) contacting a Bcl-2 family protein with the candidate compound and a peptide comprising the amino acid sequence:

| | |
|---|---|
| IAQELRRIGDEFN | [SEQ ID NO:39] |

under conditions sufficient to allow the candidate compound and the peptide to bind to the Bcl-2 family protein; and
(c) determining whether the candidate compound has bound to the Bcl-2 family protein.

In a preferred embodiment of the competitive binding assay, the candidate compound competes with a labelled peptide for binding to a Bcl-2 family member protein. The protein may be bound to a solid surface to effect separation of bound protein from the unbound labelled peptides. Alternatively, the competitive binding may be conducted in a liquid phase, and a variety of techniques may be used to detect the binding of the labelled peptides to the protein, as known in the art. The amount of bound labelled peptides may be determined to provide information on the affinity of the test compound to the Bcl-2 family protein.

Typically the screening assays described above use one or more labelled molecules. The label used in the assay can provide a detectable signal either directly or indirectly. Various labels that can be used include radioactive moieties, fluorescent compounds, chemiluminescent compounds, bioluminescent compounds and specific binding molecules. Specific binding molecules include pairs such as biotin and streptavidin, digoxin and antidigoxin etc. The binding of such labels to the peptides or proteins used in the assay may be achieved by use of standard techniques in the art.

A variety of other reagents may also be included in the reaction mixture of the assay. These include reagents such as salts, proteins, eg albumin, protease inhibitors and antimicrobial agents.

A preferred assay of the invention is to use an amplified luminescent proximity homogenous assay in which 6-His tagged (Nickel Chelate) acceptor beads and streptavidin coated donor beads allow a transfer of singlet oxygen from a donor bead to an acceptor bead when the two beads are bought into close proximity by a binding interaction.

In another aspect of the invention there is provided a method of regulating the death of a cell, comprising contacting the cell with an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, or Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence.

In another aspect of the invention there is provided a method of inducing apoptosis in unwanted or damaged cells comprising contacting said damaged or unwanted cells with an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence.

It should be understood that the cell which is treated according to a method of the present invention may be located ex vivo or in vivo. By “ex vivo” is meant that the cell has been removed from the body of a subject wherein the modulation of its activity will be initiated in vitro. For example, the cell may be a cell which is to be used as a model for studying any one or more aspects of the pathogenesis of conditions which are characterised by aberrant cell death signalling. In a preferred embodiment, the subject cell is located in vivo.

In another aspect of the invention there is provided a method of treatment and/or prophylaxis of a pro-survival Bcl-2 family member-mediated disease or condition, in a mammal, comprising administering to said mammal an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence.

In another aspect of the invention there is provided a method of treatment and/or prophylaxis of a disease or condition characterised by the inappropriate persistence or proliferation of unwanted or damaged cells in a mammal, comprising administering to said mammal an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence.

In yet another aspect of the invention there is provided a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa2 and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence, for use in a method of treatment and/or prophylaxis.

Use of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence, for regulating the death of a cell, or for inducing apoptosis in unwanted or damaged cells, or for the treatment and/or prophylaxis of a pro-survival Bcl-2 family member-mediated disease or condition, or for the treatment and/or prophylaxis of a disease or condition characterised by the inappropriate persistence or proliferation of unwanted or damaged cells.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

As used herein, the term “pro-survival Bcl-2 family member-mediated disease or condition” refers to diseases or conditions where unwanted or damaged cells are not removed by normal cellular process, or diseases or conditions in which cells undergo aberrant, unwanted or inappropriate proliferation. Such diseases include those related to inactivation of apoptosis (cell death), including disorders characterised by inappropriate cell proliferation. Disorders characterised by inappropriate cell proliferation include, for example, inflammatory conditions such as inflammation arising from acute tissue injury including, for example, acute lung injury, cancer including lymphomas, such as prostate hyperplasia, genotypic tumours, autoimmune disorders, tissue hypertrophy etc.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of conformationally constrained compounds of the invention or pharmaceutically acceptable salts or prodrugs thereof together with the subjection of the mammal to other agents or procedures which are useful in the treatment of diseases and conditions characterised by the inappropriate persistence or proliferation of unwanted or damaged cells. For example, the compounds of the present invention may be administered in combination with other chemotherapeutic drugs, or with other treatments such as radiotherapy.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that many compounds of the invention possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

The term “prodrug” is used in its broadest sense and encompasses those derivatives that are converted in vivo to the compounds of the invention. Such derivatives would readily occur to those skilled in the art, and include N-α-acyloxy amides, N-(acyloxyalkoxy carbonyl)amine derivatives and α-acyloxyalkyl esters of phenols and alcohols. A prodrug may include modifications to one or more of the functional groups of a compound of the invention.

The term “prodrug” also encompasses the use of fusion proteins or peptides comprising cell-permeant proteins or peptides and the compounds of the invention. Such fusion proteins or peptides allow the translocation of the compounds of the invention across a cellular membrane and into a cell cytoplasm or nucleus. Examples of such cell-permeant proteins and peptides include the tat peptide, membrane permeable sequences and antennapedia (penetratin), (see Dunican and Doherty, 2001).

The phrase “a derivative which is capable of being converted in vivo” as used in relation to another functional group includes all those functional groups or derivatives which upon administration into a mammal may be converted into the stated functional group. Those skilled in the art may readily determine whether a group may be capable of being converted in vivo to another functional group using routine enzymatic or animal studies.

The present invention also encompasses retro-inverso amino acid sequences. The term “retro-inverso amino acid sequence” refers to an isomer of a linear peptide in which the direction of the sequence is reversed (“retro”) and the chirality of each amino acid residue is inverted (“inverso”), Jameson et al., 1994, Brady et al., 1994. For example, if the parent peptide is Thr-Ala-Tyr, the retro modified form is Tyr-Ala-Thr, the inverso modified form is thr-ala-tyr, and the retro-inverso form is tyr-ala-thr (lower case letters refer to D-amino acids). Compared to the parent peptide, a helical retro-inverso peptide can substantially retain the original spatial conformation of the side chains but has reversed peptide bonds, resulting in a retro-inverso isomer with a topology that closely resembles the parent peptide, since all peptide backbone hydrogen bond interactions are involved in maintaining the helical structure.

While it is possible that, for use in therapy, a compound of the invention may be administered as a neat chemical, it is preferable to present the active ingredient as a pharmaceutical composition.

The invention thus further provides a pharmaceutical composition comprising a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;

each Saa is an amino acid residue with a small side chain;

Naa is an amino acid residue with a negatively charged side chain;

Xaa₁, Xaa₂, Xaa₃, Xaa₄ and Xaa₅ are each independently an amino acid residue, Zaa₁ or Zaa₂;

R is H, an N-terminal capping group or an oligopeptide optionally capped by an N-terminal capping group;

R′ is H, a C-terminal capping group or an oligopeptide optionally capped by a C-terminal capping group; and

m and n are 0 or 1, provided that at least one of m and n is 1;

wherein a conformational constraint is provided by a linker which tethers two amino acid residues, Zaa₁ and Zaa₂, in the sequence, together with one or more pharmaceutically acceptable carriers and optionally, other therapeutic and/or prophylactic ingredients. The carrier(s) must be “acceptable” in the sense of being compatible with the other ingredients of the composition and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to two hundred (200) milligrams, per tablet, are accordingly suitable representative unit dosage forms. The compounds of the present invention can be administered in a wide variety of oral and parenteral dosage forms. It will be obvious to those skilled in the art that the following dosage forms may comprise, as the active component, either a compound of the invention or a pharmaceutically acceptable salt or derivative of the compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds according to the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP).

Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 1 to 10 microns or less. Such a particle size may be obtained by means known in the art, for example by micronization.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Liquids or powders for intranasal administration, tablets or capsules for oral administration and liquids for intravenous administration are preferred compositions.

The invention will now be described with reference to the following examples which illustrate some preferred aspects of the present invention. However, it is to be understood that the particularity of the following description of the invention is not to supersede the generality of the preceding description of the invention.

### EXAMPLES

Dynamics Simulations

Molecular dynamics simulations were performed using the GROMACS v. 3.1.1 package of programs [Lindahl, 2001 #1629] with the Gromacs force field (ffgmx2). The simple point charge model for water [Berendsen, 1981 #1620] was used to describe the solvent. Ionisable amino acids were assumed to be in their standard state at neutral pH. Proteins were solvated in a cubic box of water of dimensions of 35³; no pressure coupling was applied. The total charge on the system was made neutral by replacing water molecules with sodium or chloride ions using the GENION procedure. The LINCS algorithm [Hess, 1977 #1624] was used to constrain bond lengths. Protein, water and ions were coupled separately to a thermal bath at 300 K using a Berendsen thermostat [Berendsen, 1984 #1621] applied with a coupling time of 0.1 ps. All simulations were performed using single non-bonded cut-off of 10 Å, applying a neighbour-list update frequency of 10 steps (20 fs). The particle-mesh Ewald method was applied to deal with long-range electrostatics with a grid width of 1.2 Å and a cubic interpolation scheme. All simulations consisted of an initial minimization to avoid close contacts, followed by 1 ps of ‘positional restrained’ molecular dynamics to equilibrate the water molecules (with the protein fixed). Calculations were run for a total simulation time of 50 ns using a time step of 2 fs.

Circular Dichroism

Circular dichroism spectra were obtained using a Jasco Model J-710 spetropolarimeter at 20° C. using the following parameters: path length, 2 mm; step resolution, 0.1 nm; speed, 20 nm/min; accumulation, 4; response, 1 second; bandwidth, 1.0 nm. The peptides were analysed at a concentration of 0.5 mg/mL in 30% aqueous TFE. The alpha-helical content of the peptides were determined by methods described in Yang et al (1986), involving comparisons of spectra with model helical peptides.

Peptide Synthesis

Peptides were prepared by New England Peptides, Inc, (USA) using a Pioneer peptide synthesizer or Proteomics International Pty. Ltd. (ABN 78 096 013 455; Perth, Western Australia) using an Applied Biosystems 433 peptide synthesiser using standard F-moc chemistry (Fields et al., 1991). Amino acid coupling cycles were based on the manufacturers standard protocols. Each peptide was provided with quality assurance data.

Measurement of Competition of Constrained Peptides with Bim26mer

Alphascreen (Amplified Luminsecent Proximity Homogenous Assay) is a bead based technology which measures a biological interaction between molecules. The assay consists of two hydrogel coated beads which, when bought into close proximity by a binding interaction, allow a transfer of singlet oxygen from a donor bead to an acceptor bead.

Upon binding a photosensitiser in the donor bead converts ambient oxygen to a more excited singlet state. This singlet oxygen then diffuses across to react with a chemiluminescer in the acceptor bead. Fluorophores within the same bead are activated, resulting in the emission of light.

Screening of the conformationally constrained peptides was performed using the Hexa-His detection system. Non biotinylated peptides dissolved in DMSO were titrated into the assay which consisted of 6-His tagged Bcl w delta C10 protein (24 nM Final concentration) and Biotinylated Bim BH3-26 peptide, Biotin-DLRPEIRIAQELRRIGDEFNETYTRR [SEQ ID NO: 40] (1.5 nM Final concentration). To this reaction mix 6His tagged (Nickel Chelate) acceptor beads and Streptavidin coated donor beads, both at 10 ug/ml Final concentration, were added.

Assay buffer contained 50 mM Hepes pH 7.4, 10 mM DTT, 100 mM NaCl, 0.05% Tween and 1 mg/ml BSA. Bead dilution buffer contained 50 mM Tris, pH 7.5, 0.01% Tween and 1 mg/ml BSA. The final DMSO concentration in the assay was 1%. Assays were performed in 384 well white Optiplates and analysed on the Perkin Elmer Fusion plate reader (Ex680, Em520-620 nM).

The Alphascreen 6-His detection kit and Optiplates were purchased from Perkin Elmer.

Alternatively, the detection system used was a glutathione S-transferase (GST) detection system and the assay was performed as follows:

Measurement of Competition of Constrained Peptides with Bim26mer Alphascreen (Amplified Luminsecent Proximity Homogenous Assay) is a bead based technology which measures a biological interaction between molecules. The assay consists of two hydrogel coated beads which, when bought into close proximity by a binding interaction, allow a transfer of singlet oxygen from a donor bead to an acceptor bead.

Upon binding and excitation with laser light at 680 nm a photosensitiser in the donor bead converts ambient oxygen to its excited singlet state. This singlet oxygen then diffuses across to react with a chemiluminescer in the acceptor bead. Fluorophores within the same bead are activated, resulting in the emission of light at 580-620 nm.

Screening of the conformationally constrained peptides was performed using the AlphaScreen GST (glutathione S-transferase) detection kit detection system. Non biotinylated peptides dissolved in DMSO were titrated into the assay which consisted of GST tagged Bcl w delta C29 protein (0.1 nM Final concentration) and Biotinylated Bim BH3-26 peptide, Biotin-DLRPEIRIAQELRRIGDEFNETYTRR [SEQ ID NO: 40] (3.0 nM Final concentration). To this reaction mix anti-GST coated acceptor beads and Streptavidin coated donor beads, both at 10 ug/ml Final concentration, were added and the assay mixture incubated for 4 hours at room temperature before reading.

Assay buffer contained 50 mM Hepes pH 7.4, 10 mM DTT, 100 mM NaCl, 0.05% Tween and 0.1 mg/ml casein. Bead dilution buffer contained 50 mM Tris, pH 7.5, 0.01% Tween and 0.1 mg/ml casein. The final DMSO concentration in the assay was 0.5%. Assays were performed in 384 well white Optiplates and analysed on the PerkinElmer Fusion alpha plate reader (Ex680, Em520-620 nM).

The GST Alphascreen detection kit and Optiplates were purchased from PerkinElmer.

Affinity measurements and solution competition assays (Biacore Assay).

Affinity measurements were performed on a Biacore 3000 biosensor (Biacore) with HBS (10 mM HEPES pH 7.2, 150 mM NaCl, 3.4 mM EDTA, 0.005% Tween-20,) as the running buffer. CM5 sensorchips were immobilized with mouse 26-mer wtBimBH3, and 4EBimBH3 mutant peptides using amine-coupling chemistry. To directly assess the binding affinities of pro-survival Bcl-2-like proteins for BimBH3, the proteins were directly injected into the sensorchip at 20 ml/min. After each binding measurement, residual bound protein was desorbed from the chip by injecting 50 mM Sodium Hydroxide or 6 M Guanidium Hydrochloride (pH 7.2), followed by two washes with running buffer. Binding kinetics were derived from sensorgrams, following subtraction of baseline responses, using the BIA evaluation software (version 3, Biacore). The relative affinities of BH3 peptides for pro-survival Bcl-2 proteins were assessed by comparing their abilities to compete for wtBimBH3 peptide binding to Bcl-2-like proteins. The competition binding assays were performed by incubating a fixed sub-saturating amount (10 nM) of pro-survival Bcl-2 protein with varying amounts of competitor BH3 peptide in HBS for at least 2 hr on ice. The mixtures were then injected over a sensorchip containing a channel immobilized with mouse wtBimBH3 and a control one immobilized with mouse 4EBimBH3. The baseline response (from the control channel) was subtracted to obtain the absolute binding response. Taking the response from unbound protein as the maximal response (100%), we calculated the relative residual binding (%) in the presence of increasing amounts of the competitor peptides at a given injection time point (430.5 s). The relative residual responses (f) were plotted against the initial peptide concentrations and fitted to the equation f=100/(1+(c/IC50)m), where c=concentration of the competitor peptide, m=the curvature constant, and IC50=concentration of competitor peptide required to reduce binding by 50%.

### Example 1

To investigate synthetically even a fraction of the possible linkers would be prohibitively expensive. Rather, this is a task that lends itself to prior theoretical investigation using molecular dynamics. When an adequate (eg 30 ns) simulation time is used such that several folding and unfolding events are observed, and when solvent is explicitly accounted for, molecular dynamics has been shown to be a useful predictive tool for peptide conformation (Burgi et al 2001).

Molecular dynamics simulations of length 50 nanoseconds were run on the linear Bim-like 12-mer (a) and constrained analogues (c) and (d), a 13-mer (b), and a 16-mer (e) and constrained analogues (f), (g) and (h), using explicit water, in order to see which, if any, type and position of the linker would encourage helix formation. Linkers in (c) and (f) correspond to a 1^{st} position linker as shown in formula (II) above, (d) and (g) to a 2^{nd} position constraint as shown in formula (IV) above, and (h) to a 3^{rd} position as shown in formula (VI) above, with the i(i+7) constraint corresponding to residues 94(101):

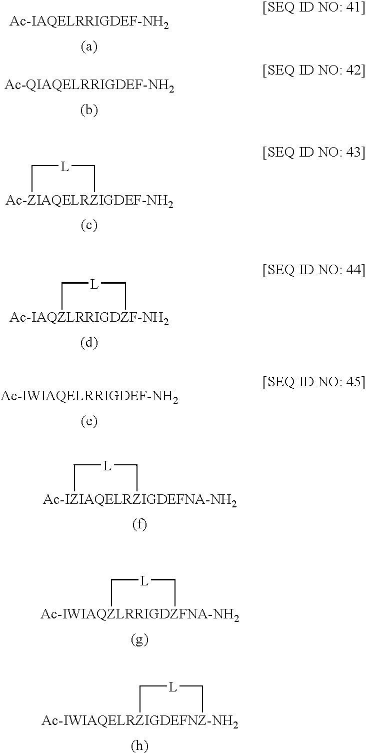

Here, Z indicates the position of the linker that connects two amino glutamic acid residues through their carboxylic acid groups. The linkers investigated were linkers —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂(═O)NH(CH₂)₂NH—, —NH(CH₂)₂NHC(═O)CH₂NH—, −NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—,

### [SEQ ID NO: 46]

NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—,

### [SEQ ID NO: 47]

—NH(CH₂)₃C(═O)NH(CH₂)₂NH— and —NH(CH₂)₂NHC(═O)(CH₂)₃NH—.

### ]SEQ ID NO: 48]

Dynamics simulations were run with the 12mer at both the 1^{st} and second positions for linkers —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, otherwise only the second position was investigated.

The dynamics simulations indicated that:

1. The unconstrained 12-mer, (a) Ac-IAQELRRIGDEF-NH₂, [SEQ ID NO: 41] was relatively helically unstable.

2. The 12-mer constrained in the 1^{st} position, (c) above, was helically a little more stable, for all linkers looked at, but tended to unravel at the C-terminus after the glycine. An exception was linker —NH(CH₂)₂S(CH₂)₂NH—, which destablized helix formation and seemed even a little worse than the linear (unconstrained) control 12-mer (a).

3. The 12-mer constrained in the 2^{nd} position, (d) above, was generally much more helical than when constrained in the 1^{st} position. In particular, the diaminopentane linker, the diaminoheptane linker, and linkers —NHCH₂C(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH— appeared to be excellent helix-stabilizing linkers. However, the diaminohexane linker, and linkers —NH(CH₂)₂S(CH₂)₂NH—, —NH(CH₂)₂SS(CH₂)₂NH—, —NH(CH₂)₂S(CH₂)₃NH—, —NH(CH₂)₂NHC(═O)(CH₂)₂NH—, —NHCH₂C(═O)NH(CH₂)₃NH—, —NH(CH₂)₃NHC(═O)CH₂NH—, —NH(CH₂)₂C(═O)NH(CH₂)₃NH—, and —NH(CH₂)₃C(═O)NH(CH₂)₂NH— were not as good at stabilizing helix formation.

4. Simulations with the 16-mer (e) generally mirrored these results.

5. The pentane linker in the 3^{rd} position of the 16-mer (h) was a little helix stabilizing, but not as good as when in the 2^{nd} position.

### Example 2

The cyclic peptide Acetyl-IAQ(E1)LRRIGD(E2)F-amide [SEQ ID NO: 41] was synthesised using Fmoc chemistry with HTBU activation on an Applied Biosystems Pioneer peptide synthesizer. The resin used during solid phase peptide synthesis was Pal-Peg-PS resin. The base peptide was prepared using orthogonal protection on the glutamic acid residues,(E1=ODMAB, O-4-{N-[1-(4,4-dimethyl-2,6-dioxocyclohexylidene)-3-methylbutyl]amino}benzyl) and (E2=O-2-PhiPR). After synthesis E2 was deprotected selectively while the peptide was still on the resin, and a 1,5-diaminopentane (mono-Fmoc protected) linker was added to the free side chain carboxyl group. Next, the Fmoc was removed, E1 was selectively deprotected and coupled to the diaminopentane linker. The remaining protecting groups and the resin were cleaved using TFA, water, and thiol based scavengers. The peptide was then purified using RP-HPLC on a C18 YMC column. MALDI-TOF DE mass spectral analysis gave M+1: 1555.

### Example 3

The peptide Ac-IAQ-E-LRRIGD-E-F-NH₂ [SEQ ID NO: 41] having a 1,6-diaminohexane linker linking the two glutamic acid residues was synthesized and purified as described in Example 2 above but using a 1,6-diaminohexane linker. MALDI-TOF DE mass spectral analysis gave M+1: 1571.

### Example 4

The peptide Ac-E-IAQELR-E-IGDEF-NH₂ [SEQ ID NO: 49] having a 1,5-diaminopentane linker linking the two glutamic acid residues was synthesized and purified as described in Example 2 above. MALDI-TOF DE mass spectral analysis gave M+1: 1657.

### Example 5

The preparation of linker precursor NH₂CH₂CC(═O)NHCH₂CH₂NH-Fmoc was synthesized from commercially available compounds Fmoc-NH(CH₂)₂NH₂.HCl (1.9g 6 mmol) and t-Boc-Gly-Osu (1.6 g, 6 mmol), were dissolved in DMF (15 mL), then treated with N-ethyl-N,N-diisopropylamine (2.1 mL, 12 mmol) and stirred for 2 hours. Water (40 mL) was added to precipitate the product, t-Boc-NH₂CH₂C(═O)NHCH₂CH₂NH-Fmoc, a colourless powder after filtering and air-drying. This was then dissolved in 4M HCl/Ether (15 mL) and stood for 2 hours. The supernatant was decanted and the remaining while granules washed with ether, filtered and dried, giving the product HCl.NH₂CH₂C(═O)NHCH₂CH₂NH-Fmoc in 33% overall yield for the two steps. MS (m/z'340). 1H NMR (300 MHz, DMSO) δ: 8.51 (broad triplet, 1H, NH); 8.14 (broad singlet, 3H, NH₃); 7.3-7.9 (multiplet, 8H+1H, ArH (Fmoc)+NH); 4.15-4.35 (multiplet, 3H, CH₂CH (Fmoc)); 3.49, (singlet, 2H, CH₂ (gly)); 3.15 (triplet, 2H, CH₂); 3.05 (triplet, 2H, CH₂). Chemical shift (δ) are measured in parts per million (ppm).

### Example 6

The peptide Ac-IAQ-E-LRRIGD-E-F-NH₂ [SEQ ID NO: 41] having a —NHCH₂C(═O)NHCH₂CH₂NH— linker linking the two glutamic acid residues was synthesized analogously to Example 2 but using the mono-Fmoc protected linker described in Example 6, except that E1 was selectively deprotected first and reacted with the mono-Fmoc protected linker. The Fmoc was then removed and E2 was deprotected and coupled to the linker.

### Example 7

Four constrained peptides were synthesized as described in Examples 2 to 6, corresponding to the pentane linker in the first position (A), the pentane linker in the second position (B), the hexane linker in the second position (C), and linker —NHCH₂C(═O)NH(CH₂)₂NH— in the second position (D).

Their circular dichroism spectra were measured as a gauge of their helicity in 30% aqueous trifluoroethanol (TFA), and their affinity to Bcl-2 ΔC22, Bcl-w ΔC10 and Bcl-w-ΔC29 measured by means of a competition assay using biotinylated Bim-BH3 peptide. The results are shown below:

| | | IC₅₀ (nM) | IC₅₀ (nM) | IC₅₀ (nM) |
|---|---|---|---|---|
| Peptide | % Helicity | Bcl-2 ΔC22 | Bcl-w ΔC10 | Bcl-w ΔC29 |
| Linear 12mer | 9 | 240,000 | 870 | 4,700 |
| A | 33 | 26,000 | 2,600 | 1,800 |
| B | 28 | 290 | 65 | 150 |
| C | 39 | 2,600 | 230 | 120 |
| D | 16 | 6,900 | 40 | 160 |

The circular dichroism spectra indicated that the constrained peptides were in general more helical—some much more so—than the linear 12-mer. Peptides B and C displayed outstanding increases in affinity for Bcl-2 and Bcl-xL over the unconstrained 12-mer. These sorts of peptides form the basis of the current claim.

### Example 8

The linear 16-mer peptide based on the Bim BH3-only protein, Ac-IWIAQELRRIGDEFNA-NH₂ [SEQ ID NO: 50] was prepared using a Pioneer Peptide Synthesizer and purified by HPLC. The constrained peptides were synthesized as described in Examples 2 to 6. The first constrained peptide (E) has a pentane linker tethering the two glutamate residues. The second constrained peptide (F) has a —NHCH₂C(═O)(CH₂)₂NH₂— linker tethering the two glutamic acid residues.

The affinity of linear 16-mer and peptides (E) and (F) for Bcl-w ΔC29 was measured by means of a competition assay using biotinylated BIM-BH3 peptide. The results are shown below.

| | IC50 (nM) | Mass Spectrometry |
|---|---|---|
| Peptide | Bcl-w ΔC29 | MW |
| linear 16-mer | 2.5 | 1972 |
| E | 0.5 | 2037 |
| F | 0.3 | 2054 |

The constrained 16-mer peptides had improved binding affinity with Bcl-w ΔC29.

### Example 9

To ascertain the effect of specific residues in the sequence on binding to Bcl-w ΔC29, substitutions were made in the sequence and IC₅₀ values measured. The peptides used, with the exception of Peptide G, were linear peptides synthesized on a Pioneer peptide synthesiser or Applied Biosystems 433 Peptide Synthesiser using standard F-moc chemistry, Fields et al. (1991). Amino acid coupling cycles were based on the manufacturers standard protocols. Peptide G is a constrained peptide which has a pentane linker between the two glutamic acid residues and was prepared as described in Examples 2 to 6.

| | |
|---|---|
| Mass | IC₅₀ nM |
| Spectrometry | Bcl-w |

| | | | | |
|---|---|---|---|---|
| Peptide | Sequence | SEQ ID | MW | ΔC29 |

| | | | | |
|---|---|---|---|---|
| linear 16-mer | Ac-IWIAQELRRIGDEFNA-NH₂ | [SEQ ID NO:50] | 1972 | 2.5 |
| G | Ac-QAIAQZLRRIGDZFNA-NH₂ | [SEQ ID NO:51] | 1940 | 2.4 |
| (constrained) | | | | |
| H (linear) | Ac-IWIAQQLRRIGDQFNA-NH₂ | [SEQ ID NO:52] | 1969 | 3.3 |
| I (linear) | Ac-IWAAQELRRIGDEFNA-NH₂ | [SEQ ID NO:53] | 1930 | 360 |
| J (linear) | Ac-IWIAQEARRIGDEFNA-NH₂ | [SEQ ID NO:54] | 1930 | 3700 |
| K (linear) | Ac-IWIAQELRRAGDEFNA-NH₂ | [SEQ ID NO:55] | 1930 | 7.3 |
| L (linear) | Ac-IWIAQELRRIGDEANA-NH₂ | [SEQ ID NO:56] | 1896 | 3500 |
| M (linear) | Ac-IWAAQEARRAGDEANA-NH₂ | [SEQ ID NO:57] | 1836 | 64,000 |
| N (linear) | Ac-IFIAQELRRIGDEFNA-NH₂ | [SEQ ID NO:58] | 1933 | 11 |
| O (linear) | Ac-AWIAQELRRIGDEFNA-NH₂ | [SEQ ID NO:59] | 1930 | 22 |
| P (linear) | Ac-IAIAQELRRIGDEFNA-NH₂ | [SEQ ID NO:60] | 1857 | 42 |
| Q (linear) | Ac-IRIAQELRRIGDEFNA-NH₂ | [SEQ ID NO:61] | 1942 | 17 |
| R (linear) | Ac-IWIAQELRRIGDEFAN-NH₂ | [SEQ ID NO:62] | 1972 | 12 |
| S (linear) | Ac-IWIAQELRRIGDEFAA-NH₂ | [SEQ ID NO:63] | 1929 | 3.3 |
| T (linear) | Ac-IWIAQELCitCitIGDEFNA-NH₂ | [SEQ ID NO:64] | 1975 | 20 |
| U (linear) | Ac-IWIAQELRRIGDEFNN-NH₂ | [SEQ ID NO:65] | 2015 | 5.8 |

Replacement of the first two residues in the constrained peptide (G) with the helix stabilizing QA residues led to a reduction in binding of the constrained peptide (E:0.5 nM, G:2.4 nM), indicating that one or both of the I and W residues interacts favourably with the Bcl-w protein.

The importance of the first two residues I and W can also be seen in the linear peptides. When W→F (peptide N), I→A (peptide O), W→A (peptide P) and W→R (peptide Q) substitutions are made, there is also a drop in binding compared to the linear 16-mer.

To confirm that it was the constraint that provided increased binding activity and not just the loss of two negative charges in the sequence, the two glutamate residues were amidated to provide glutamine residues (peptide I). This resulted in a slight decrease in binding affinity, not an increase.

To show the importance of the hydrophobic residues, each Haa was substituted with alanine. Peptide I (I→A) showed a 100-fold decrease in binding affinity, Peptide J (L→A) showed about 1000-fold decrease in affinity, Peptide K (I→A) showed a 3-fold decrease in affinity and Peptide L (F→A) showed a 1,000-fold decrease in affinity. When all 4 Haa were substituted by alanine there was a 25,000-fold decrease in binding affinity.

Peptide S, Peptide T and Peptide U are substitutions at the last two residues in the sequence. Peptide S (NA→AA) showed only slight, if any, loss of binding affinity, while Peptide U (NA→NN) showed about a two-fold loss. However, when both residues were substituted (by reversal, NA→AN), these losses were more than additive and there is a 4-5-fold decrease in affinity.

### Example 10

Two further peptides related to Puma and Bmf BH3-only proteins were synthesized on a Pioneer peptide synthesizer and their binding affinity for Bcl-2 ΔC26 assessed.

| | |
|---|---|
| Mass | IC₅₀ nM |
| Spectrometry | Bcl-w |

| | | | | |
|---|---|---|---|---|
| Peptide | Sequence | SEQ ID | MW | ΔC29 |

| | | | | |
|---|---|---|---|---|
| Puma | Ac-REIGAQLRRMADDLNA-NH₂ | [SEQ ID NO:66] | 1870 | 52 |
| Bmf | Ac-VQIARKLQAIADQFHR-NH₂ | [SEQ ID NO:67] | 1935 | 0.25 |

### Example 11

Bcl-w has been used in Examples 8 to 10 because it is a robust protein to use. However as shown below, when tested for affinity to Bcl-2 AC22, Bcl-w ΔC10 and Bcl-w ΔC29 using the Biacore assay and Bcl-w ΔC29 using the Alpha screen assay with GST detection, the Bim-26mer shows similar potency with respect to Bcl-w and Bcl-2. In line with the results shown in example 7, constrained peptides will also potently inhibit the binding of Bim26mer to Bcl-2 and more so than their linear counterparts.

IC₅₀ nM

| | | | |
|---|---|---|---|
| IC₅₀ nM | IC₅₀ nM | IC₅₀ nM | Bcl-w |
| Bcl-w | Bcl-w | Bcl-w | ΔC29 |
| ΔC29 | ΔC22 | ΔC10 | Alpha |

| | | | | | |
|---|---|---|---|---|---|
| Peptide | Sequence | Biacore | Biacore | Biacore | Screen |

| | | | | | |
|---|---|---|---|---|---|
| hsBimL/Bod | DMRPEIWIAQELRR | 4.3 | 2.6 | 6 | 0.1 |
| (81-106) | IGDEFNAYYARR | | | | |
| | [SEQ ID NO:68] | | | | |

### Example 12

A retro inverso peptide having the sequence

[SEQ ID NO:69]

Ac-a-n-f-e-d-g-i-r-r-l-e-q-a-i-w-i-NH₂

(Small letters refer to D-amino acids), was synthesised on an Applied Biosystems 433 Peptide Synthesiser using standard F-moc chemistry, Fields et al. (1991). Amino acid coupling cycles were based on manufacturers standard protocols. The peptide was purified by HPLC and molecular weight by mass spectrometry was 1971.

### Example 13

Cell Based:

The efficacy of the compounds of the present invention can also be determined in cell based killing assays using a variety of cell lines and mouse tumour models. For example, their activity on cell viability can be assessed on a panel of cultured tumorigenic and non-tumorigenic cell lines, as well as primary mouse or human cell populations, e.g. lymphocytes. Cell viability and total cell numbers can be monitored over 3-7 days of incubation with 1 nM-100 μM of the compounds to identify those that kill at IC50<10 μM.

The compounds of the present invention can also be evaluated for the specificity of their targets and mode of action in vivo. For example, if a compound binds with high selectivity to Bcl-2, it should not kill cells lacking Bcl-2. Hence, the specificity of action can be confirmed by comparing the activity of the compound in wild-type cells with those lacking Bcl-2, derived from Bcl-2-deficient mice.

### Example 14

Animal Models:

To assess the anti-tumour efficacy of the compounds of the present invention in vivo, the BH3 mimetics can be given alone (intra-venously; iv or intra-peritoneally; ip) or in combination with sub-optimal doses of clinically relevant chemotherapy (e.g. 25-100 mg/kg cyclophospahmide intra-peritoneally). Mice injected intra-peritoneally with 10⁶ Bcl-2-overexpressing mouse lymphoma cells (Strasser 1996; Adams 1999) develop an aggressive immature lymphoma that is rapidly fatal within 4 weeks if untreated, but are partially responsive to cyclophosphamide. The lymphoma/leukaemia can readily be monitored by performing peripheral blood counts in the animals using a Coulter counter or by weighing the lymphoid organs (lymph nodes, spleen) when the animals are sacrificed. Another model is implantation of a cell line such as that derived from human follicular lymphoma (DoHH2) into immunocompromised SCID mice (Lapidot 1997). Because the compounds of the invention are contemplated to be efficacious in combination therapy, their in vivo activity can be evaluated alone or in combination with conventional chemotherapeutic agents (e.g. cyclophosphamide, doxorubucin, epipodophylotoxin (etoposide; VP-16)). Cohorts of 18-20 mice per treatment arm will be studied to enable a 25% difference in efficacy with a power of 0.8 at a significance level of 0.05 to be determined. These in vivo tests in mice will also generate preliminary pharmacokinetic, pharmacodynamic and toxicology data.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Adams J M and Cory S, Oncogene cooperation in leukemogenesis. Cancer Surveys 15:119-141, 1992.
Adams J M and Cory S, The Bcl-2 protein family: arbiters of cell survival. Science 281: 1322-1326, 1998.
Adams, J. M., A. W. Harris, A. Strasser, S. Ogilvy, and S. Cory. Transgenic models of lymphoid neoplasia and development of a pan-hematopoietic vector. Oncogene 18, 5268, 1999.
Adams J M and Cory S, Life-or-death decisions by the Bcl-2 protein family. Trends in Biochemical Sciences 26: 61-66, 2001.
Antonsson B, Montessuit S, Sanchez B and Martinou J C, Bax is present as a high molecular weight oligomer/complex in the mitochondrial membrane of apoptotic cells. Journal of Biological Chemistry 276(15): 11615-11623., 2001
Ashkenazi A and Dixit V M, Death receptors: signaling and modulation. Science 281(5381): 1305-1308, 1998.
Bouillet P, Metcalf D, Huang D C S, Tarlinton D M, Kay T W H, Kontgen F, Adams J M and Strasser A, Proapoptotic Bcl-2 relative Bim required for certain apoptotic responses, leukocyte homeostasis, and to preclude autoimmunity. Science 286: 1735-1738, 1999.
Bouillet P, Cory S, Zhang L-C Strasser A and Adams J M, Degenerative disorders caused by Bcl-2 deficiency are prevented by loss of its BH3-only antagonist Bim. Developmental Cell 1(5): 645-653, 2001.
Brady L and Dodson G, Reflections on a peptide, Nature, 368 (6473), 692-693, 1994.
Brown J M and Wouters B G, Apoptosis, p53 and tumor cell sensitivity to anticancer agents. Cancer Research 59 (7): 1391-1399, 1999.
Burgi R, Daura X, Mark A, Bellanda M, Mammi S, Peggion E, van Gunsteren W; Folding study of an Aib-rich peptide in DMSO by molecular dynamics simulations, Journal of Peptide Research, 57, 107-118, 2001.
Chen F, Hersh B M, Conradt B, Zhou Z, Riemer D, Gruenbaum Y and Horvitz H R, Translocation of C. elegans CED-4 to nuclear membranes during programmed cell death. Science 287(5457): 1485-1489, 2000.
Cheng E H, Wei M C, Weiler S, Flavell R A, Mak T W, Lindsten T and Korsmeyer S J, Bcl-2, Bcl-x_{L} sequester BH3 domain-only molecules preventing BAX and BAK-mediated mitochondrial apoptosis. Molecular Cell 8(3): 705-711, 2001.
Chinnaiyan A M, O'Rourke K, Lane B R and Dixit V M, Interaction of CED-4 with CED-3 and CED-9: a molecular framework for cell death. Science 275(5303): 1122-1126, 1997.
Cleary M L, Smith S D and Sklar J, Cloning and structural analysis of cDNAs for Bcl-2 and a hybrid Bcl-2/immunoglobulin transcript resulting from the t(14;18) translocation. Cell 47(1): 19-28, 1986.
Conradt B and Horvitz H R, The C. elegans protein EGL-1 is required for programmed cell death and interacts with the Bcl-2-like protein CED-9. Cell 93(4): 519-529, 1998.
Conus S, Rosse T and Borner C, Failure of Bcl-2 family members to interact with Apaf-1 in normal and apoptotic cells. Cell Death and Differentiation, 7(10): 947-954, 2000.
Corey S and Adams J M, The Bcl-2 family: Regulators of the cellular life-or-death switch; Nature Reviews Cancer, 2, 647-656, 2002.
Corey S and Adams J M, Apoptosomes: engines for caspase activation; Current Opinion in Cell Biology; 14 (6): 715-720, 2002.
Desagher S, Osen-Sand A, Nichols A, Eskes R, Montessuit S, Lauper S, Maundrell K, Antonsson B and Martinou J-C, Bid-induced conformational change of Bax is responsible for mitochondrial cytochrome c release during apoptosis. Journal of Cell Biology 144(5): 891-901,1999.
Du C, Fang M, Li Y, Li L and Wang X, Smac, a mitochondrial protein that promotes cytochrome c-dependent caspase activation by eliminating IAP inhibition. Cell 102(1): 33-42, 2000.
Dunican D J and Doherty P; Designing Cell-permeant phosphopeptides to modulate intracellular signalling pathways; Biopolymers (Peptide Science), 60, 45-60, 2001.
Eskes R, Desagher S, Antonsson B and Martinou J C, Bid induces the oligomerisation and insertion of Bax into the outer mitochondrial membrane. Molecular and Cellular Biology 2(3): 929-935, 2000.
Fields C G, Lloyd D H, Macdonald R L, Otteson K M, Noble R L, HBTU activation for automated Fmoc solid-phase peptide synthesis, Peptide Research, 4(2), 95-101, 1991.
Fisher D E, Apoptosis in cancer therapy: crossing the threshold. Cell 78: 539-542, 1994.
Green T. W and Wutz P, Protective Groups in Organic Synthesis, John Wiley & Son, 3^{rd} Ed., 1999.
Han J, Flemington C, Houghton A B, Gu Z, Zambetti G P, Lutz R J, Zhu L and Chittenden T, Expression of bbc2, a pro-apoptotic BH3-only gene, is regulated by diverse cell death and survival signals. Proceedings of the National Academy of Sciences of the USA 98(20): 11318-11323, 2001.
Haraguchi M, Torii S, Matsuzawa S, Xie Z, Kitada S, Krajewski S, Yoshida H, Mak T W and Reed J C, Apoptotic protease activating factor 1 (Apaf-1)-independent cell death suppression by Bcl-2. Journal of Experimental Medicine 191(10): 1709-1720, 2000.
Hausmann G, O'Rielly L A, van Driel R, Beaumont J G, Strasser A, Adams J M and Huang D C S and Strasser A, BH3-only proteins—essential initiators of apoptotic cell death. Cell 103: 839-842, 2000.
Jameson, B A, McDonnell J M, Marini J C, Korngold R, A rationally designed CD4 analogue inhibits experimental allergic encephalomyelitis, Nature, 368(6473), 744-746, 1994.
Jürgensmeier J M, Xie Z, Deveraux Q, Ellerby L, Bredesen D and Reed J C, Bax directly induces release of cytochrome c from isolated mitochondria. Proceedings of the National Academy of Sciences of the United States of America 95(9): 4997-5002, 1998.
Kamada S, Shimono A, Shinto Y, Tsujimura T, Takahashi T, Noda T, Kitamura Y, Kondoh H and Tsujimoto Y, Bcl-2 deficiency in mice leads to pleiotropic abormalities: accelerated lymphoid cell death in thymus and spleen, polycystic kidney, hair hypopigmentation, and distorted small intestine. Cancer Research 55: 354-359, 1995.
Kluck R M, Bossy-Wetzel E, Green D R and Newmeyer D D, The release of cytochrome c from mitochondria—a primary site for Bcl-2 regulation of apoptosis. Science275(5303): 1132-1136, 1997.
Korsmeyer S J, Wei M C, Saito M, Weiler S, Oh K J and Schlesinger P H, Pro-apoptotic cascade activates BID, which oligomerizes BAK or BAX into pores that result in the release of cytochrome c. Cell Death and Differentiation 7(12): 1166-1173, 2000.
Lane D P, p53, guardian of the genome. Nature 358: 15-16, 1992.
Lapidot, T., Y. Fajerman, and O. Kollet. Immune-deficient SCID and NOD/SCID mice models as functional assays for studying normal and malignant human hematopoiesis. Journal of Molecular Medicine 75, 664, 1997.
Li P, Nijhawan D, Budihardjo I, Srinivasula S M, Ahmad M, Alnermri E S and Wang X, Cytochrome c and dATP-dependent formation of Apaf-1(Caspase-9 complex initiates an apoptic protease cascade. Cell 91: 479-489, 1997.
Lowe S W, Schmitt E M, Smith S W, Osborne B A and Jacks T, p53 is required for radiation-induced apoptosis in mouse thymocyctes. Nature 362: 847-849, 1993.
Marsden V S, O'Connor L, O'Reilly L A, Silke J, Metcalf D, Ekert P G, Huang D C, Cecconi F, Kuida K, Tomaselli K J, Roy S, Nicholson D W, Vaux D L, Bouillet P, Adams J M and Strasser A; Apoptosis initiated by Bcl-2-regulated caspase activation independently of the cytochrome c/Apaf-1/caspase-9 apoptosome; Nature, 419(6907) 634-7, 2002.
Mikhailov V, Mikhailova M, Pulkrabek D J, Dong Z, Venkatachalam M A and Saikumar P, Bcl-2, prevents bax oligomerization in the mitochondrial outer membrane. Journal of Biological Chemistry 20: 20, 2001.
Moriishi K, Huang D C S, Cory S and Adams J M, Bcl-2 family members do not inhibit apoptosis by binding the caspase-activator Apaf-1. Proceedings of the National Academy of Sciences of the United States of America 96: 9683-9688, 1999.
Motoyama N, Wang F P, Roth K A, Sawa H, Nakayama K, Nakayama K, Negishi I, Senju S, Zhang Q, Fujii S and Loh D Y, Massive cell death of immature hematopoietic cells and neurons in Bcl-x deficient mice. Science 267: 1506-1510, 1995.
Motoyama N, Kimura T, Takahashi T, Watanabe T and Nakano T, Bcl-x prevents apoptotic cell death of both primitive and definitive erythrocytes at the end of maturation. Journal of Experimental Medicine 189(11): 1691-1698, 1999.
Nakano K and Wousden K H, PUMA, a novel proapoptotic gene, is induced by p53. Molecular Cell 7:683-694, 2001.
Nakayama K, Nakayama K-I, Negishi I, Kuida K, Sawa H and Loh D Y, Targeted disruption of Bcl-2ab in mice: occurrence of gray hair, polycystic kidney disease, and lymphocytopenia. Proceedings of the National Academy of Sciences of the United States of America 91: 3700-3704, 1994.
Oda E, Ohki R, Murasawa H, Nemoto J, Shibue T, Yamashita T, Tokino T, Taniguchi T and Tanaka N, Noxa, a BH3-only member of the Bcl-2 family and candidate mediator of p53-induced apoptosis. Science 288(5468): 1053-1058, 2000.
Parrish J, Metters H, Chen L and Xue D, Demonstration of the in vivo interaction of key cell death regulators by structure-based design of second-site suppressors. Proceedings of the National Academy of Sciences of the USA 97(22): 11916-11921, 2000.
Petros A M, Nettesheim D G, Wang Y, Olejniczak E T, Meadows R P, Mack J, Swift K, Matayoshi E D, Zhang H, Thompson C B and Fesik S W, Rationale for Bcl-x_{L}/Bad peptide complex formation from structure, mutagenesis and biophysical studies. Protein Science 9: 2528-2534, 2000.
Print C G, Loveland K L, Gibson L, Meehan T, Stylianou A, Wreford N, de Kretser D, Metcalf D, Köntgen F, Adams J M and Cory S, Apoptosis regulator Bcl-w is essential for spermatogenesis but appears otherwise redundant. Proceedings of the National Academy of Sciences of the United States of America 95: 12424-12431, 1998.
Puthalakath H, Huang D C S, O'Reilly L A, King S M and Strasser A, The proapoptotic activity of the Bc1-2 family member Bim is regulated by interaction with the dynein motor complex. Molecular Cell 3: 287-296, 1999.
Puthalakath H, Villunger A, O'Reilly L A, Beaumont J G, Coultas L, Cheney R E, Huang D C S and Strasser A, Bmf: a pro-apoptotic BH3-only protein regulated by interaction with the myosin V actin motor complex, activated by anoikis. Science 293: 1829-1832, 2001.
Roberge et al., Science, 269: 202-204, 1995.
Ross A J, Waymire K G, Moss J E, Parlow A F, Skinner M K, Russell L D and MacGregor G R, Testicular degeneration in Bclw-deficient mice. Nature Genetics 18(3): 251-256, 1998.
Sambrook J. et al. Molecular Cloning: A Laboratory Manual, 2^{nd} Edition, Cold Spring Harbor Laboratory Press, Plainview, N.Y., 1989.
Sattler M, Liang H, Nettesheim D, Meadows R P, Harlan J E, Eberstadt M, Yoon H S, Shuker S B, Chang B S, Minn A J, Thompson C B and Fesik S W, Structure of Bcl-x_{L}-Bak peptide complex: recognition between regulators of apoptosis. Science 275(5302):983-986, 1997.
Spector M S, Desnoyers S, Hoeppner D J and Hengartner M O, Interaction between the C. elegans cell-death regulators CED-9 and CED-4. Nature 385(6617): 653-656, 1997.
Strasser, Harris A W, Bath M L and Cory S, Novel primitive lymphoid tumours induced in transgenic mice by cooperation between myc and Bcl-2. Nature 348:331-333, 1990.
Strasser A, Harris A W, Jacks T and Cory S, DNA damage can induce apoptosis in proliferating lymphoid cells via p53-independent mechanisms inhibitable by Bcl-2. Cell 79:329-339, 1994.
Strasser, A., A. G. Elefanty, A. W. Harris, and S. Cory. Progenitor tumours from Em-bcl-2-myc transgenic mice have lymphomyeloid differentiation potential and reveal developmental differences in cell survival. EMBO Journal 15, 3823, 1996.
Strasser A, Huang D C S and Vaux D L, The role of the Bcl-2/CED-9 gene family in cancer and general implications of defects in cell death control for tumourigenesis and resistance to chemotherapy. Biochimica et Biophysica Acta 1333:F151-F178, 1997.
Strasser A, O'Connor L and Dixit V M, Apoptosis signaling. Annual Review of Biochemistry 69: 217-245, 2000.
Suzuki M, Youle R J and Tjandra N, Structure of Bax: coregulation of dimer formation and intracellular localization. Cell 103: 645-654, 2001.
Thompson C B, Apoptosis in the pathogenesis and treatment of disease. Science 267(5203): 1456-1462, 1995.
Thomberry N A and Lazebnik Y, Caspases: enemies within. Science, 281 (5381): 1312-1316, 1998.
Tsujimoto Y, Cossman J, Jaffe E and Croce C M, Involvement of the Bcl-2 gene in human follicular lymphoma. Science 228(4706): 1440-1443, 1985.
Vaux D L, Cory S and Adams J M, Bcl-2 gene promotes haemopoietic cell survival and cooperates with c-myc to immortalize pre-B cells. Nature 335: 440-442, 1988.
Veis D J, Sorenson C M, Shutter J R and Korsmeyer S J, Bcl-2-deficient mice demonstrate fulminant lymphoid apoptosis, polycystic kidneys, and hypopigmented hair. Cell 75: 229-240, 1993.
Verhagen A M, Ekert P G, Pakusch M, Silke J, L.M. C, Reid G E, Moritz R L, Simpson R J and Vaux D L, Identification of DIABLO, a mammalian protein that promotes apoptosis by binding to and antagonizing inhibitor of apoptosis (IAP) proteins. Cell 102(1): 43-53, 2000.
Vousden K H, p53; death star. Cell 103: 691-694, 2000.
Wang K, Yin X-M, Chao D T, Milliman C L and Korsmeyer S J, BID: a novel BH3 domain-only death agonist. Genese and Development 10: 2859-2869, 1996.
Wei M C, Zong W X, Cheng E H, Lindsten T, Panoutsakopoulou V, Ross A J, Roth K A, MacGregor G R, Thompson C B and Korsmeyer S J, Proapoptotic BAX and BAK: a requiesite gateway to mitochondrial dysfunction and death. Science 292(5517): 727-730, 2001.
Wu D Y, Wallen H D and Nunez G, Interaction and regulation of subcellular localization of CED-4 and CED-9. Science 275(5303): 1126-1129, 1997.
Yang J T, Wu C S C and Martinez H G; Calculation of protein conformation by circular dichroism; Methods Enzymol., 130, 208-269, 1986.
Yang J, Liu X S, Bhalla K, Kim C N, Ibrado A M, Cai J Y, Peng T-I, Jones D P and Wang X D, Prevention of apoptosis by Bc1-2—release of cytochrome c from mitochondria blocked. Science 275(5303): 1129-1132, 1997.
Yang X, Chang H Y and Baltimore D, Essential role of CED-4 oligomerization in CED-3 activation and apoptosis. Science 281(5381): 1355-1357, 1998.
Yu J, Zhang L, Hwang P M, Kinzler K W and Vogelstein B, PUMA induces the rapid apoptosis of colorectal cancer cells. Molecular Cell 7: 673-682, 2001.
Zong W X, Lindsten T, Ross A J, MacGregor G R and Thompson C B, BH3-only proteins that bind pro-survival Bcl-2 family members fail to induce apoptosis in the absence of Bax and Bak. Genes and Development 15(12): 1481-1486, 2001.
Zou H, Henzel W J, Liu X, Lutschg A and Wang X, Apaf-1, a human protein homologous to C. elegans CED-4, particpates in cytochrome c-dependent activation of Caspase-3. Cell 90: 405-4134, 1997.

1. A conformationally constrained compound or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa4 is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group or an oligopeptide optionally capped by an N-terminal capping group;
R′ is H, a C-terminal capping group or an oligopeptide optionally capped by a C-terminal capping group; and
m and n are 0 or 1, provided that at least one of m and n is 1;
wherein a conformational constraint is provided by a linker (L) which tethers two amino acid residues, Zaa₁ and Zaa₂, in the sequence.

2. A conformationally constrained compound or pharmaceutically acceptable salt or prodrug thereof according to claim 1 wherein all of Haa₁, Haa₂, Haa₃ and Haa₄ are amino acid residues with a hydrophobic side chain.

3. A conformationally constrained compound or pharmaceutically acceptable salt or prodrug thereof according to claim 1 wherein Haa₁, Haa₂, Haa₃ and Haa₄ are independently selected from L-phenylalanine, L-isoleucine, L-leucine, L-valine, L-methionine and L-tyrosine.

4. A conformationally constrained compound or pharmaceutically acceptable salt or prodrug thereof according to claim 1 wherein Haa₂ is L-leucine.

5. A conformationally constrained compound or pharmaceutically acceptable salt or prodrug thereof according to claim 1 wherein each Saa is independently selected from glycine, L-alanine, L-serine, L-cysteine and aminoisobutyric acid.

6. A conformationally constrained compound or pharmaceutically acceptable salt or prodrug thereof according to claim 1 wherein Naa is an L-aspartic acid or an L-glutamic acid residue.

7. A conformationally constrained compound or pharmaceutically acceptable salt or prodrug thereof according to claim 1 wherein R is an N-terminal capping group or an oligopeptide having 1 to 10 amino acid residues selected from Xaa₁, optionally capped with an N-terminal capping group.

8. A conformationally constrained compound or pharmaceutically acceptable salt or prodrug thereof according to claim 7 wherein R is an N-terminal capping group selected from acyl and N-succinate.

9. A conformationally constrained compound or pharmaceutically acceptable salt or prodrug thereof according to claim 1 wherein R′ is a C-terminal capping group or an oligopeptide having 1 to 10 amino acid residues selected from Xaa₁, optionally capped with a C-terminal capping group.

10. A conformationally constrained compound or pharmaceutically acceptable salt or prodrug thereof according to claim 9, wherein the C-terminal capping group is NH₂.

11. A conformationally constrained compound or pharmaceutically acceptable salt or prodrug thereof according to claim 1, wherein Xaa₁, Xaa₂, Xaa₃, Xaa₄ and Xaa₅ are independently selected from L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine and L-valine.

12. A conformationally constrained compound or pharmaceutically acceptable salt or prodrug thereof according to claim 1 wherein the linker (L) tethers two non-adjacent amino acids in an i(i+7) relationship where the first end of the linker is attached to a first amino acid residue (Zaa₁) at a first position and the other end of the linker is attached to a second amino acid residue (Zaa₂) which is positioned 7 amino acids after Zaa₁.

13. A conformationally constrained compound or pharmaceutically acceptable salt or prodrug thereof according to claim 1 wherein L is 4 to 8 atoms in length.

14. A conformationally constrained compound or pharmaceutically acceptable salt or prodrug thereof according to claim 12 wherein Zaa₁ is located before Haa₁ at the N-terminal of the sequence and Zaa₂ is located between Haa₂ and Haa₃.

15. A conformationally constrained compound or pharmaceutically acceptable salt or prodrug thereof according to claim 12 wherein Zaa₁ is located between Haa₁ and Haa₂ and Zaa₂ is located between Haa₃ and Haa₄.

16. A conformationally constrained compound or pharmaceutically acceptable salt or prodrug thereof according to claim 12 wherein Zaa₁ is located between Haa₂ and Haa₃ and Zaa₂ is located after Haa₄ at the C-terminal end of the amino acid sequence.

17. A conformationally constrained compound or pharmaceutically acceptable salt or prodrug thereof according to claim 1 wherein Zaa₁ and Zaa₂ are independently selected from L-aspartic acid, L-glutamic acid, L-lysine, L-ornithine, D-aspartic acid, D-glutamic acid, D-lysine, D-ornithine, L-β-homoaspartic acid, L-β-homoglutamic acid, L-β-homolysine, L-α-methylaspartic acid, L-α-methylglutamic acid, L-α-methyllysine, L-α-methylornithine, D-α-methylaspartic acid, D-α-methylglutamic acid, D-α-methyllysine and L-α-methylomithine.

18. A conformationally constrained compound or pharmaceutically acceptable salt or prodrug thereof according to claim 17 wherein Zaa₁ and Zaa₂ are independently selected from L-aspartic acid, L-glutamic acid, L-lysine and L-ornithine.

19. A conformationally constrained compound or pharmaceutically acceptable salt or prodrug thereof according to claim 18 wherein Zaa₁ and Zaa₂ are independently selected from L-aspartic acid and L-glutamic acid.

20. A conformationally constrained compound or pharmaceutically acceptable salt or prodrug thereof according to claim 1 wherein Zaa₁ and Zaa₂ have side chains containing a carboxylic acid and the linker is selected from the group consisting of —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)₂N⁺H₂(CH₂)₂NH—, —NH(CH₂)₂S(CH₂)₂NH—, —NHCH₂C(═O)NH(CH₂)₂NH—, —NH(CH₂)₂NHC(═O)CH₂NH—, —NH(CH₂)₂SS(CH₂)₂—NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—.

21. A conformationally constrained compound or pharmaceutically acceptable salt or prodrug thereof according to claim 20 wherein the linker is selected from the group consisting of —NH(CH₂)₅NH—, —NH(CH₂)₆NH—, —NH(CH₂)₇NH—, —NHCH₂C(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—.

22. A conformationally constrained compound or pharmaceutically acceptable salt or prodrug thereof according to claim 20 wherein the linker is selected from the group consisting of —NH(CH₂)₅NH— and —NHCH₂C(═O)NH(CH₂)₂NH—.

23. A conformationally constrained compound or pharmaceutically acceptable salt or prodrug thereof according to claim 1 wherein Zaa₁ and Zaa₂ have side chains containing an amino group and the linker is selected from the group consisting of —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂—C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH2)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—.

24. A conformationally constrained compound or pharmaceutically acceptable salt or prodrug thereof according to claim 23 wherein the linker is selected from the group consisting of —C(═O)(CH₂)₅C(═O)—, —C(═O)(CH₂)₆C(═O)—, —C(═O)(CH₂)₇C(═O)—, —C(═O)CH₂C(═O)NH(CH₂)₂C(═O)—, —C(═O)(CH₂)₂NHC(═O)CH₂C(═O)—, —C(═O)(CH₂)₂O(CH₂)₃C(═O)— and —C(═O)(CH₂)₂C(═O)NH(CH₂)₂C(═O)—.

25. A conformationally constrained compound or pharmaceutically acceptable salt or prodrug thereof according to claim 23 wherein the linker is selected from the group consisting of —C(═O)(CH₂)₅C(═O)— and —C(═O)CH₂C(═O)NH(CH₂)₂C(═O)—.

26. A conformationally constrained compound or pharmaceutically acceptable salt or prodrug thereof according to claim 1 wherein Zaa₁ has a side chain containing an amino group and Zaa₂ has a side chain containing a carboxylic acid and the linker is selected —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂—NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(═O)(CH₂)₂C(═O)NH(CH₂)₃NH—, —C(═O)(CH2)₃NHC(═O)(CH₂)₂NH—, —C(═O)(CH₂)₃C(═O)NH(CH₂)₂NH— and —C(═O)(CH₂)₂NHC(═O)(CH₂)₃NH—.

27. A conformationally constrained compound or pharmaceutically acceptable salt or prodrug thereof according to claim 26 wherein the linker is selected from the group consisting of —C(═O)(CH₂)₅NH—, —C(═O)(CH₂)₆NH—, —C(═O)(CH₂)₇NH—, —C(═O)CH₂C(═O)NH(CH₂)₂NH—, —C(═O)(CH₂)₂NHC(═O)CH₂NH—, —C(═O)(CH₂)₂O(CH₂)₃NH—and —C(═O)(CH₂)₂C(═O)NH(CH₂)₂NH—.

28. A conformationally constrained compound or pharmaceutically acceptable salt or prodrug thereof according to claim 26 wherein the linker is selected from the group consisting of —C(═O)(CH₂)₅NH— and —C(═O)CH₂C(═O)NH(CH₂)₂NH—.

29. A conformationally constrained compound or pharmaceutically acceptable salt or prodrug thereof according to claim 1 wherein Zaa₁ has a side chain containing a carboxylic acid and Zaa₂ has a side chain containing an amino group and the linker is selected from the group consisting of —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)—, —NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)—.

30. A conformationally constrained compound or pharmaceutically acceptable salt or prodrug thereof according to claim 29 wherein the linker is selected from the group consisting of —NH(CH₂)₅C(═O)—, —NH(CH₂)₆C(═O)—, —NH(CH₂)₇C(═O)—, —NHCH₂C(═O)NH(CH₂)₂C(═O)—, —NH(CH₂)₂NHC(═O)CH₂C(═O)—, —NH(CH₂)₂0(CH₂)₃C(═O)— and —NH(CH₂)₂C(═O)NH(CH₂)₂C(═O)—.

31. A conformationally constrained compound or pharmaceutically acceptable salt or prodrug thereof according to claim 29 wherein the linker is selected from the group consisting of —NH(CH₂)₅C(═O)— and —NHCH₂C(═O)NH(CH₂)₂C(═O)—.

32. A conformationally constrained compound or pharmaceutically acceptable salt or prodrug thereof according to claim 1, of any one of formulae (II) to (VI): 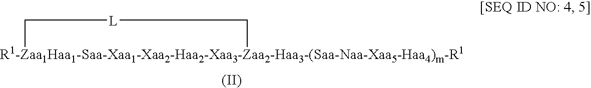
wherein Haa₁, Haa₂, Haa₃, Haa₄, Xaa₁, Xaa₂, Xaa₃, Xaa₅, Saa, Naa and L are as defined above for formula (I), m is 0 or 1, R¹ and R^{1′} are as defined above for R and R′ in formula (I), Zaa₁-L-Zaa₂ represents two amino acid residues with their side chains bridged by a linker L; 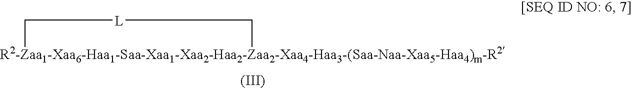
wherein Haa₁, Haa₂, Haa₃, Haa₄, Xaa₁, Xaa₂, Xaa₄, Xaa₅, Saa, Naa and L are as defined above for formula (I), Xaa₆ is an amino acid residue as defined for Xaa₁ above; m is 0 or 1, R² and R^{2′} are as defined above for R and R′ in formula (I), Zaa₁-L-Zaa₂ represents two amino acid residues with their side chains bridged by a linker L; 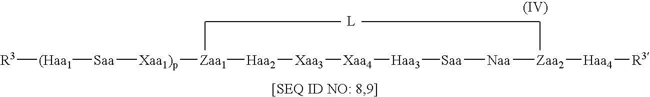
wherein Haa₁, Haa₂, Haa₃, Haa₄, Xaa₁, Xaa₃, Xaa₄, Saa, Naa and L are as defined above for formula (I), p is 0 or 1, R³ and R^{3′} are as defined above for R and R′ in formula (I), Zaa₁-L-Zaa₂ represents two amino acid residues with their side chains bridged by a linker L; 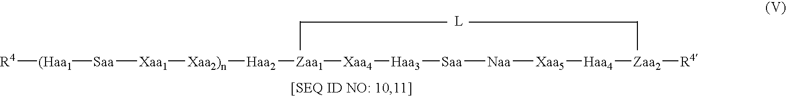
wherein Haa₁, Haa₂, Haa₃, Haa₄, Xaa₁, Xaa₂, Xaa₄, Xaa₅, Saa, Naa and L are as defined above in formula (I), n is 0 or 1, R⁴ and R^{4′} are as defined above for R and R′ in formula (I), Zaa₁-L-Zaa₂ represents two amino acid residues with their side chains bridged by a linker L; and 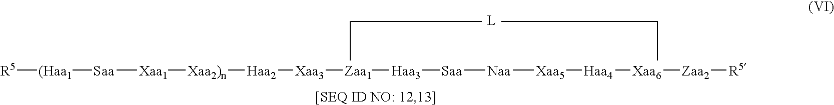
wherein Haa₁, Haa₂, Haa₃, Haa₄, Xaa₁, Xaa₂, Xaa₃, Xaa₅, Saa, Naa and L are as defined above for formula (I), Xaa₆ is an amino acid residue as defined for Xaa₁ above; n is 0 or 1, R⁵ and R^{5′} are as defined above for R and R′ in formula (I), Zaa₁-L-Zaa₂ represents two amino acid residues with their side chains bridged by a linker L; or a pharmaceutically acceptable salt or prodrug thereof.

33. A conformationally constrained compound or pharmaceutically acceptable salt or prodrug thereof according to claim 32 having structural formula (VII): 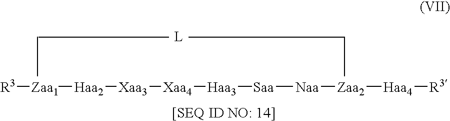
wherein Zaa₁, Haa₂, Xaa₃, Xaa₄, Haa₃, Saa, Naa, Zaa₂, Haa₄, R³, R^{3′} and L are defined above in formula (IV).

34. A conformationally constrained compound or pharmaceutically acceptable salt or prodrug thereof according to claim 1 having structural formula (VIII): 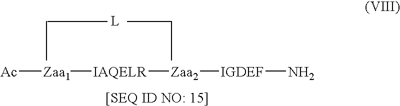
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH— and —NH(CH₂)₂NHC(═O)(CH₂)₂NH—; or
where Zaa₁ and Zaa₂ are selected from L-lysine and ornithine; and
L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂₃C(═O)—, —C(═O)(CH₂)₂C(═O)NH(CH₂)₂C(═O)— and —C(═O)(CH₂)₂NHC(═O)(CH₂)₂C(═O)—; or where Zaa₁ is selected from L-aspartic acid, L-glutamic acid and Zaa₂ is selected from L-lysine and ornithine; and
L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₃C(═O)—, —NH(CH₂)₂C(═O)NH(CH₂)₂C(═O)— and —NH(CH₂)₂NHC(═O)(CH₂)₂C(═O)—; or
where Zaa₁ is selected from L-lysine and ornithine and Zaa₂ is selected from L-aspartic acid, L-glutamic acid; and
L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH— and —C(═O)(CH₂)₂NHC(═O)(CH₂)₂NH—.

35. A conformationally constrained compound or pharmaceutically acceptable salt or prodrug thereof according to claim 1 having structural formula (IX): 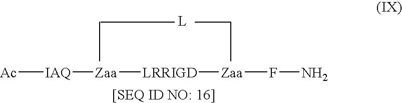
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—; or
where Zaa₁ and Zaa₂ are selected from L-lysine and ornithine; and
L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N^{+H}₂(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH₂)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—; or
where Zaa₁ is selected from L-aspartic acid, L-glutamic acid and Zaa₂ is selected from L-lysine and ornithine; and
L is selected from —NH(CH₂)₄C(═O)—, —NH(CH₂)SC(═O)—, —NH(CH₂)₆C(═O)—, —NH(CH₂)₇C(═O)—, —NH(CH₂)₂O(CH₂)₂C(═O)—, —NH(CH₂)N+H₂(CH₂)₂C(═O)—, —NH(CH₂)S(CH₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)——NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)— and —NH(CH₂)₂NHC(═O)(CH₂)₃C(═O)—; or
where Zaa₁ is selected from L-lysine and ornithine and Zaa₂ is selected from L-aspartic acid, L-glutamic acid; and
L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N+H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(═O)(CH₂)₂C(═O)NH(CH₂)₃NH—, —C(═O)(CH₂)₃NHC(═O)(CH₂)₂NH—, —C(═O)(CH₂)₃C(═O)NH(CH₂)₂NH— and —C(═O)(CH₂)₂NHC(═O)(CH₂)₃NH—.

36. A conformationally constrained compound or pharmaceutically acceptable salt or prodrug thereof according to claim 1 selected from the group consisting of: 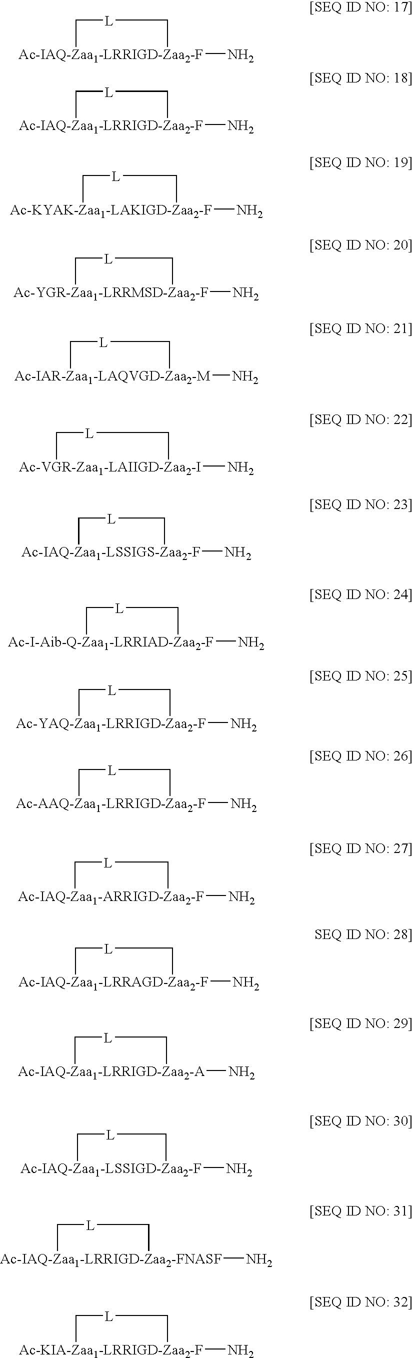
wherein Zaa₁ and Zaa₂ are as defined in claim 17 and L is a linker which tethers Zaa₁ and Zaa₂.

37. A conformationally constrained compound or pharmaceutically acceptable salt or prodrug thereof according to claim 36 wherein Zaa₁ and Zaa₂ are independently selected from L-aspartic acid and L-glutamic acid and L is selected from the group consisting of —NH(CH₂)₅NH—, —NH(CH₂)₆NH—, —NH(CH₂)₇NH—, —NHCH₂(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—.

38. A conformationally constrained compound or pharmaceutically acceptable salt or prodrug thereof according to claim 37 wherein L is selected from the group consisting of —NH(CH₂)₅NH— and —NHCH₂C(═O)NH(CH₂)₂NH—.

39. A conformationally constrained compound or pharmaceutically acceptable salt or prodrug thereof according to claim 1 selected from the group consisting of: 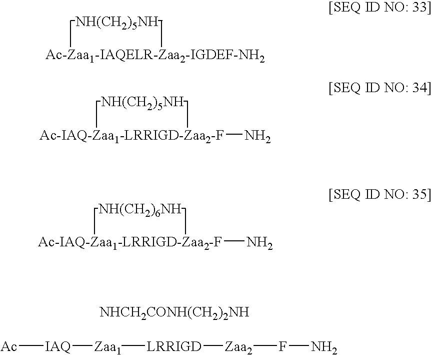
wherein Zaa₁ and Zaa₂ are independently selected from L-aspartic acid and L-glutamic acid.

40. A conformationally constrained compound or pharmaceutically acceptable salt or prodrug thereof according to claim 39 wherein Zaa₁ and Zaa₂ are both L-glutamic acid.

41. A pharmaceutical composition comprising a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group or an oligopeptide optionally capped by an N-terminal capping group;
R′ is H, a C-terminal capping group or an oligopeptide optionally capped by a C-terminal capping group; and
m and n are 0 or 1, provided that at least one of m and n is 1;
wherein a conformational constraint is provided by a linker which tethers two amino acid residues, Zaa₁ and Zaa₂, in the sequence, together with one or more pharmaceutically acceptable carriers and optionally, other therapeutic and/or prophylactic ingredients.

42. An assay for identifying compounds which bind to a member of the Bcl-2 family of proteins, the assay comprising the steps of:
(a) providing a candidate compound to be tested;
(b) contacting a Bcl-2 family protein with the candidate compound and a peptide comprising the amino acid sequence:

| | |
|---|---|
| IAQELRRIGDEFN | [SEQ ID NO:37] |

under conditions sufficient to allow the candidate compound and the peptide to bind to the Bcl-2 family protein; and
(c) determining whether the candidate compound has bound to the Bcl-2 family protein.

43. An assay according to claim 42 wherein the peptide has an amino acid sequence:

| | |
|---|---|
| DLRPEIRIAQELRRIGDEFNETYTRR. | [SEQ ID NO:38] |

44. A method of regulating the death of a cell, comprising contacting the cell with an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group or an oligopeptide optionally capped by an N-terminal capping group;
R′ is H, a C-terminal capping group or an oligopeptide optionally capped by a C-terminal capping group; and
m and n are 0 or 1, provided that at least one of m and n is 1;
wherein a conformational constraint is provided by a linker which tethers two amino acid residues, Zaa₁ and Zaa₂, in the sequence.

45. A method of inducing apoptosis in unwanted or damaged cells comprising contacting said damaged or unwanted cells with an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group or an oligopeptide optionally capped by an N-terminal capping group;
R′ is H, a C-terminal capping group or an oligopeptide optionally capped by a C-terminal capping group; and
m and n are 0 or 1, provided that at least one of m and n is 1;
wherein a conformational constraint is provided by a linker which tethers two amino acid residues, Zaa₁ and Zaa₂, in the sequence.

46. A method of treatment and/or prophylaxis of a pro-survival Bcl-2 family member-mediated disease or condition, in a mammal, comprising administering to said mammal an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group or an oligopeptide optionally capped by an N-terminal capping group;
R′ is H, a C-terminal capping group or an oligopeptide optionally capped by a C-terminal capping group; and
m and n are 0 or 1, provided that at least one of m and n is 1;
wherein a conformational constraint is provided by a linker which tethers two amino acid residues, Zaa₁ and Zaa₂, in the sequence.

47. A method according to claim 46 wherein the disease or condition is an inflammatory condition, a cancer or an autoimmune disorder.

48. A method of treatment and/or prophylaxis of a disease or condition characterised by the inappropriate persistence or proliferation of unwanted or damaged cells in a mammal, comprising administering to said mammal an effective amount of a conformationally constrained compound, or a pharmaceutically acceptable salt or prodrug thereof, the compound comprising an amino acid sequence (I):

| | | |
|---|---|---|
| (I) | R-(Haa₁-Saa-Xaa₁-Xaa₂)ₙ-Haa₂- | [SEQ ID NO:1-3] |
| | Xaa₃-Xaa₄-Haa₃-(Saa-Naa-Xaa₅- | |
| | Haa₄)ₘ-R′ | |

wherein Haa₁, Haa₂, Haa₃ and Haa₄ are each independently an amino acid residue with a hydrophobic side chain or when n and m are both 1, one of Haa₁, Haa2 and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group or an oligopeptide optionally capped by an N-terminal capping group;
R′ is H, a C-terminal capping group or an oligopeptide optionally capped by a C-terminal capping group; and
m and n are 0 or 1, provided that at least one of m and n is 1;
wherein a conformational constraint is provided by a linker which tethers two amino acid residues, Zaa₁ and Zaa₂, in the sequence.
